(19) United States
(12) Patent Application Publication
(10) Pub. No.: US 2023/0172920 A1
(43) Pub. Date: Jun. 8, 2023

(54) DOSING REGIMEN FOR TREATING A DISEASE MODULATED BY CSF-1R

(71) Applicant: Novartis AG, Basel (CH)

(72) Inventors: Astrid JULLION, Sierentz (FR); Christophe MEILLE, Michelbach le haut (FR); Cornelia QUADT, Allschwil (CH); Michael John ROY, Madison, NJ (US); Marion WIESMANN, Bottmingen (CH)

(21) Appl. No.: 17/996,616

(22) PCT Filed: Apr. 19, 2021
(86) PCT No.: PCT/IB2021/053198

§ 371 Date: Oct. 19, 2022

Related U.S. Application Data
(60) Provisional application No. 63/013,151, filed on Apr. 21, 2020.
(60) Provisional application No. 63/145,780, filed on Feb. 4, 2021.

(51) Int. Cl. A61K 31/4439; A61P 25/28

(52) U.S. Cl. CPC A61K 31/4439; A61P 25/28

(57) ABSTRACT

The present disclosure relates to the field of pharmacy, particularly to a CSF-1R inhibitor for use in the treatment of disease modulated by CSF-1R. For example, the disclosure relates to a CSF-1R inhibitor for use in the treatment of cancer or neurodegenerative diseases. The disclosure also relates to a CSF-1R inhibitor or a pharmaceutical combination comprising a CSF-1R inhibitor, or a pharmaceutically acceptable salt thereof, and an anti-PD-1 antibody molecule, or a pharmaceutically acceptable salt thereof, for use in the treatment of cancer; to a method for the treatment of cancer that involves administering a CSF-1R inhibitor or the combination; and to the use of a CSF-1R inhibitor or the combination for the manufacture of a medicament for the treatment of cancer.

### SEQUENCE LISTING

The instant application contains a Sequence Listing which has been submitted electronically in ASCII format and is hereby incorporated by reference in its entirety. Said ASCII copy, created on Feb. 15, 2021, is named PAT058798-WO-PCT_SL.txt and is 87,285 bytes in size.

### FIELD OF THE DISCLOSURE

The present disclosure relates to the field of pharmacy, particularly to a CSF-1R inhibitor for use in the treatment of disease modulated by CSF-1R. For example, the disclosure relates to a CSF-1R inhibitor for use in the treatment of cancer or neurodegenerative diseases. The disclosure also relates to a CSF-1R inhibitor or a pharmaceutical combination comprising a CSF-1R inhibitor, or a pharmaceutically acceptable salt thereof, and an anti-PD-1 antibody molecule, or a pharmaceutically acceptable salt thereof, for use in the treatment of cancer; to a method for the treatment of cancer that involves administering a CSF-1R inhibitor or the combination; and to the use of a CSF-1R inhibitor or the combination for the manufacture of a medicament for the treatment of cancer.

### BACKGROUND

CSF-1R is the receptor for M-CSF (macrophage colony stimulating factor, also called CSF-1) and mediates the biological effects of this cytokine. The cloning of the colony stimulating factor-1 receptor (also called c-fms) was described for the first time in Roussel et al., Nature 325:549-552 (1987). In that publication, it was shown that CSF-1R had transforming potential dependent on changes in the C-terminal tail of the protein including the loss of the inhibitory tyrosine 969 phosphorylation which binds CbI and thereby regulates receptor down regulation (Lee et al., EMBO 18:3616-28 (1999)).

CSF-1R is a receptor tyrosine kinase (RTK) and a member of the family of immunoglobulin (Ig) motif containing RTKs characterized by repeated Ig domains in the extracellular portion of the receptor. The intracellular protein tyrosine kinase domain is interrupted by a unique insert domain that is also present in the other related RTK class III family members that include the platelet derived growth factor receptors (PDGFR), stem cell growth factor receptor (c-Kit) and fms like cytokine receptor (FLT3). In spite of the structural homology among this family of growth factor receptors, they have distinct tissue-specific functions.

The main biological effects of CSF-1R signaling are the differentiation, proliferation, migration and survival of the precursor macrophages and osteoclasts from the monocytic lineage. Activation of CSF-1R is mediated by its ligand, CSF-1. Binding of CSF-1 to CSF-1R induces the formation of homodimers and activation of the kinase by tyrosine phosphorylation. Further signaling is mediated by the p85 subunit of PI3K and Grb2 connecting to the PI3K/AKT and Ras/MAPK pathways, respectively. These two important signaling pathways can regulate proliferation, survival and apoptosis. Other signaling molecules that bind the phosphorylated intracellular domain of CSF-1R include STAT1, STAT3, PLCγ and CbI (see Bourette & Rohrschneider, Growth Factors 17:155-166 (2000)).

The knockout animals for either CSF-1 (op/op mouse; see Pollard et al., Adv Devel Biochem 4:153-193 (1996)) or CSF-1R (Dai X M et al., Blood 99(1):111-20 (2002)) have osteopetrotic, hematopoietic, tissue macrophage, and reproductive phenotypes consistent with a role for CSF-1R in these cell types.

There are three distinct mechanisms by which CSF-1R signaling is likely involved in tumor growth and metastasis. The first is that expression of CSF-ligand and receptor has been found in tumor cells originating in the female reproductive system (breast, ovarian, endometrium) (Scholl J., Nat. Can. Inst. 94:120-126 (1994); Kacinsky et al., Mol. Reprod. Dev. 46:71-74 (1997)), and the expression has been associated with breast cancer xenograft growth as well as poor prognosis in breast cancer patients. Two point mutations were seen in CSF-1R in about 10-20% of acute myelocytic leukemia, chronic myelocytic leukemia and myelodysplasia patients tested in one study, and one of mutations was found to disrupt receptor turnover (Rigde et al., PNAS 87:1377-1380 (1990)). Mutations were also found in some cases of hepatocellular cancer (Yang et al., Hepatobiliary Pancreat Dis Int 3:86-9 (2004)) and idiopathic myelofibrosis (Abu Duhier et al., Br J Haematol 120:464-70 (2003)).

The second mechanism is based on blocking signaling through M-CSF/CSF-1R at metastatic sites in bone, which, when inhibited, reduces osteoclastogenesis, bone resorption and osteolytic bone lesions. Breast, kidney, and lung cancers have been found to metastasize to the bone and cause osteolytic bone disease resulting in skeletal complications. Inhibition of CSF-1R kinase activity in osteoclasts with a small molecule inhibitor is likely to prevent these skeletal related events in metastatic disease.

The third mechanism is based on the recent observation that tumor associated macrophages (TAM) found in solid tumors of the breast, prostate, ovarian and cervical cancers correlated with poor prognosis (Bingle et al., J Pathol 196(3):254-65 (2002)). Macrophages are recruited to the tumor by M-CSF and other chemokines. The macrophages can then contribute via TAMs to tumor progression through the secretion of angiogenic factors, proteases and other growth factors and may be blocked by inhibition of CSF-1R signaling. On the other hand macrophages are known to have a tumoricidal effect through phagocytosis and direct cytotoxicity. The specific role of macrophages with respect to the tumor still needs to be better understood including the potential spatial and temporal dependence on their function and the relevance to specific tumor types.

Cancers of the brain and nervous system are among the most difficult to treat. Prognosis for patients with these cancers depends on the type and location of the tumor as well as its stage of development. For many types of brain cancer, average life expectancy after symptom onset may be months or a year or two. Treatment consists primarily of surgical removal and radiation therapy; chemotherapy is also used, but the range of suitable chemotherapeutic agents is limited, perhaps because most therapeutic agents do not penetrate the blood-brain barrier adequately to treat brain tumors. Using known chemotherapeutics along with surgery and radiation rarely extends survival much beyond that produced by surgery and radiation alone. Thus improved therapeutic options are needed for brain tumors.

Gliomas are a common type of brain tumor. They arise from the supportive neuronal tissue comprised of glial cells (hence the name glioma), which maintain the position and function of neurons. Gliomas are classified according to the type of glial cells they resemble: astrocytomas (including glioblastomas) resemble star-shaped astrocyte glial cells, oligodendrogliomas resemble oligodendrocyte glial cells; and ependymomas resemble ependymal glial cells that form the lining of fluid cavities in the brain. In some cases, a tumor may contain a mixture of these cell types, and would be referred to as a mixed glioma.

The typical current treatment for brain cancers is surgical removal of the majority of the tumor tissue, which may be done by invasive surgery or using biopsy or extractive methods. Gliomas tend to disseminate irregularly, though, and are very difficult to remove completely. As a result, recurrence nearly always occurs soon after tumor removal. Radiation therapy and/or chemotherapy can be used in combination with surgical removal, but these generally provide only modest extension of survival time. For example, recent statistics showed that only about half of patients in the U.S. who are diagnosed with glioblastoma are alive one year after diagnosis, and only about 25% are still alive after two years, even when treated with the current standard of care combination treatments.

Glioblastoma multiforme (GBM) is the most common adult primary brain tumor and is notorious for its lethality and lack of responsiveness to current treatment approaches. Unfortunately, there have been no substantial improvements in treatment options in recent years, and minimal improvements in the survival prospects for patients with GBM. Thus there remains an urgent need for improved treatments for cancers of the brain such as gliomas.

Gliomas develop in a complex tissue microenvironment comprised of many different types of cells in the brain parenchyma in addition to the cancer cells themselves. Tumor-associated macrophages (TAMs) are one of the prominent stromal cell types present, and often account for a substantial portion of the cells in the tumor tissues. Their origin is not certain: these TAMs may originate either from microglia, the resident macrophage population in the brain, or they may be recruited from the periphery.

TAMs can modulate tumor initiation and progression in a tissue-specific manner: they appear to suppress cancer development in some cases, but they enhance tumor progression in the majority of studies to date. Indeed, in approximately 80% of the cancers in which there is increased macrophage infiltration, the elevated TAM levels are associated with more aggressive disease and poor patient prognosis. Several studies have shown that human gliomas also exhibit a significant increase in TAM numbers, which correlates with advanced tumor grade, and TAMs are typically the predominant immune cell type in gliomas. However, the function of TAMs in gliomagenesis remains poorly understood, and it is currently not known whether targeting of these cells represents a viable therapeutic strategy. In fact, opposing effects on tumor growth have been reported in the literature, in some cases even where a similar experimental strategy was used to deplete macrophages in the same orthotopic glioma implantation model. In some studies, TNF-α or integrin β3 produced by TAMs have been implicated in the suppression of glioma growth, whereas in other reports CCL2 and MT1-MMP have been proposed as enhancers of tumor development and invasion.

Inhibition of CSF-1R signaling represents a novel, translationally relevant approach that has been used in several oncological contexts, including xenograft intratibial bone tumors. However, it has not yet been shown to be effective in brain tumors. Some non-brain cancers have been targeted with compounds that affect a variety of cell types that are associated with, or support, tumor cells rather than directly targeting the tumor cells themselves. For example. PLX3397 is reported to co-inhibit three targets (FMS, Kit, and Flt3-ITD) and to down-modulate various cell types including macrophages, microglia, osteoclasts, and mast cells. PLX3397 has been tested for treating Hodgkin's lymphoma. However, Hodgkin's lymphoma responds well to various chemotherapeutics, according to the PLX3397 literature, while brain tumors are much more resistant to chemotherapeutics and have not been successfully treated. As demonstrated herein, a CSF-1R inhibitor had no direct effect on proliferation of glioblastoma cells in culture, though, and it did not reduce numbers of macrophage cells in tumors of treated animals. It is thus surprising that, as also demonstrated herein, a CSF-1R inhibitor can effectively inhibit growth of brain tumors in vivo, cause reduction in tumor volume in advanced stage GBM, and even apparently eradicating some glioblastomas.

The Programmed Death 1 (PD-1) protein is an inhibitory member of the extended CD28/CTLA4 family of T-cell regulators (Okazaki et al. Curr. Opin. Immunol. 2002, 14, 391779; Bennett et al. J. Immunol. 2003, 170, 711). Ligands of the CD28 receptor include a group of related B7 molecules, also known as the “B7 Superfamily” (Coyle et al. Nature Immunol. 2001, 2(3), 203; Sharpe et al. Nature Rev. Immunol. 2002, 2, 116; Collins et al. Genome Biol. 2005, 6, 223.1; Korman et al Adv. Immunol. 2007, 90, 297). Several members of the B7 Superfamily are known, including 27.1 (CD80), 27.2 (CD86), the inducible co-stimulator ligand (ICOS-L), the programmed death-1 ligand (PD-L1; 27-H1), the programmed death-2 ligand (PD-L2; B7-DC), B7-H3, S7-H4 and B7-H6 (Collins et al. Genome Biol. 2005, 6, 223.1). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

PD-L1 is abundant in a variety of human cancers (Dong et al Nat. Med. 2002, 8, 787). PD-1 is known as an immune-inhibitory protein that negatively regulates TCR signals (Ishida et al. EMBO J. 1992, 11, 3887; Blank et al. Immunol. Immunother. 2006, 56(5), 739). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. J. Mol. Med. 2003, 81, 281; Blank et al. Cancer Immunol. Immunother. 2005, 54, 307; Konishi et al. Clin. Cancer Res. 2004, 10, 5094). Immune suppression can be reversed by inhibiting the local interaction of PD-1 with PD-L1 or PD-L2; the effect is additive when the interaction of PD-1 with PD-L2 is blocked as well (Iwai et al. Proc. Nat. Acad. Sci. USA 2002, 99:12293-7; Brown et al. J. Immunol. 2003, 170, 1257).

The CSF-1R inhibitor BLZ945 has been investigated as single agent and in combination with spartalizumab in a phase I clinical study. During the course of the clinical study, adverse events (AEs) of all grades and regardless of relationship to study treatment were reported in all patients treated with single agent BLZ945 and in all patients treated with the combination regimen. The most frequently reported AEs (>10%) suspected to be related to BLZ945 single agent were increased aspartate aminotransferase (AST), nausea, vomiting, increased alanine aminotransferase (ALT), fatigue, increased amylase, increased blood creatine phosphokinase and decreased appetite. The most frequently reported suspected AEs (>10%) with the combination treatment were increased AST, increased ALT, pruritus, fatigue, nausea, rash and vomiting. While the increased levels of ALT/AST by BLZ945 are considered to be caused by reduced clearance of these enzymes due to inhibition of Kupffer cells in the liver, such observations are typically viewed as indications of liver damage. Therefore, there exists an unmet medical need for alternative dosing regimens of the compounds of Formula (I) that will result in delivering a therapeutically effective dose with minimal adverse events.

Separately, BLZ945 is being investigated in amyotrophic lateral sclerosis (ALS). ALS is a fatal degenerative disease characterized by a progressive loss of the upper motor neurons (UMNs) in the motor cortex and lower motor neurons (LMNs) at the spinal or bulbar level [Rowland L P and Schneider N A, 2001]. Given the median survival (3-5 years), the rapid decline in functional measures and the poor prognosis in the natural course of the disease, the burden of ALS upon patients, family members and caregivers is substantial. The incidence of ALS is about 5000/year in the USA, with approximately 16000 prevalent patients in the US and 29000 in EU (Logroscino G, and Piccininni M.)

The mainstay of care for patients with ALS is timely intervention to manage symptoms, including use of nasogastric feeding, prevention of aspiration, and provision of ventilatory support (usually with bi-level positive airway pressure) (R. H. Brown and al. 2017). Currently, the available therapies offer limited clinical benefit for patients with ALS. Riluzole and edaravone, have been approved in the US; however, there remains an urgent unmet medical need for therapies that have the potential to slow the progression of ALS.

Neuroinflammation plays a key role in ALS (Rodriguez and Mahy 2016). An important pathophysiological mechanism in ALS is microglial activation that is associated with degenerative motor neurons. Recent evidence links macrophage colony stimulating factor 1 receptor (CSF-1R) dependent microgliosis with ALS disease progression (Martinez Muriana et al 2016). A highly selective and potent CSF-1R inhibitor has the potential to treat ALS patients by slowing disease progression by targeting microglia cells, thereby delaying the requirement for mechanical ventilation, improving quality of life and increasing survival in patients with ALS. BLZ945 is a potent and selective inhibitor of CSF-1R. In preclinical studies using a rodent model of ALS, BLZ945 demonstrated a benefit on the maintenance of normal body weight gain as well as a dose-dependent delay of disease-related impairments in grip strength and muscle innervation.

This efficacy was associated at necropsy with dose-dependent depletion of microglia from the spinal cord. In patients with ALS, a recent Phase 2b/3 clinical trial with masitinib, a CSF-1R inhibitor of modest potency and low specificity, demonstrated significant delay of disease progression. Taken together, these data suggest that selective CSF-1R inhibition using BLZ945 to deplete microglia may represent a therapeutic strategy for preventing or slowing ALS progression. However, an increase in ALT/AST by treatment with BLZ945 is ALS patients, will greatly impact the dosing regimen that is safe and efficacious for treating ALS patients. Therefore, there remains a need for a safe and efficacious treatment for ALS patients. Therefore, selective inhibition of CSF-1R to deplete microglia with a specific dosing regimen is expected to represent a therapeutic strategy for preventing or slowing ALS progression. Such a selective CSF-1R inhibitor would also allow co-administration with other interventional approaches, for example, in combination with the current standard of care, riluzole or edaravone, or other ALS clinical compounds.

### SUMMARY OF EMBODIMENTS OF THE INVENTION

The present invention is based on demonstrations that advanced solid tumors can be treated with an inhibitor of CSF-1R. The effectiveness of the CSF-1R inhibitors described herein is believed to be due to their inhibition of certain activities of TAMs, even though it does not appear to significantly reduce the number of TAMs present, and is likely also a function of the demonstrated ability of these compounds to penetrate the blood-brain barrier effectively in subjects with a brain tumor. These methods provide much needed new therapeutic options for patients diagnosed with advanced solid tumors, particularly brain tumors, particularly glioblastomas.

Colony stimulating factor-1 (CSF-1), also termed macrophage colony stimulating factor (M-CSF), signals through its receptor CSF-1R (also known as c-FMS) to regulate the differentiation, proliferation, recruitment and survival of macrophages. Small molecule inhibitors of CSF-1R have been developed that block receptor phosphorylation by competing for ATP binding in the active site, as for other receptor tyrosine kinase inhibitors. The present invention uses a potent, selective CSF-1R inhibitor, which penetrates the blood-brain barrier (BBB), to block CSF-1R signaling in glioma as illustrated in the RCAS-PDGF-B-HA/Nestin-Tv-a; Ink4a/Arf^{-/-} mouse model of gliomagenesis. This genetically engineered glioma model is ideal for preclinical testing as a model for human GBM, as it recapitulates all features of human GBM in an immunocompetent setting. Because it closely models human GEM, and proneural GBM in particular, efficacy in this model is expected to translate into clinical efficacy on human glioblastomas such as glioblastoma multiforme and mixed gliomas.

The invention can be practiced with any inhibitor of CSF-1R capable of penetrating the brain. Some such compounds are the 6-O-substituted benzoxazole and benzothiazole compounds disclosed in WO2007/1214B4, particularly the compounds of Formula IIa and IIb in that reference, and the compounds disclosed herein.

In one aspect, the invention provides a CSF-1R inhibitor of Formula (I) 4-((2-(((1R,2R)-2-hydroxycyclohexyl)amino)benzo[d]thiazol-6-yl)oxy)-N-methylpicolinamide:

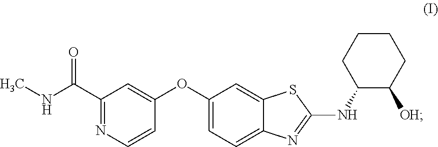 [SMILES: CNC(=O)c1cc(Oc2ccc3nc(N[C@@H]4CCCC[C@H]4O)sc3c2)ccn1]

or a pharmaceutically acceptable salt thereof, for use in the treatment of a CSF-1R modulated disease, such as cancer or neurodegenerative diseases, wherein (I) is administered 4 days-on and 10 days-off, twice per cycle.

In another aspect, the invention provides a pharmaceutical combination comprising (I) a CSF-1R inhibitor of formula (I) 4-((2-(((1R,2R)-2-hydroxycyclohexyl)amino)benzo[d]thiazol-6-yl)oxy)-N-methylpicolinamide [Compound of Formula (I)]:

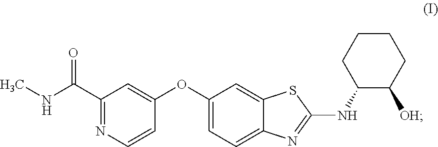 [SMILES: CNC(=O)c1cc(Oc2ccc3nc(N[C@@H]4CCCC[C@H]4O)sc3c2)ccn1]

or a pharmaceutically acceptable salt thereof and (ii) an anti-PD-1 antibody molecule or a pharmaceutically acceptable salt thereof, for use in the treatment of cancer, wherein (i) is administered 4 days-on and 10 days-off twice per cycle and (ii) is administered at least once per cycle.

### BRIEF DESCRIPTION OF THE DRAWINGS

FIG. 1: Simulated Kinetics of ALT (and AST), based on pre-clinical data, suggest a need for a washout period

FIG. 2: Cynomolgus monkey model based on pre-clinical data depicts that switching to 4-days on/10-days off may reduce max ALT and return to baseline level

FIG. 3: Preliminary efficacy data comparing BLZ945 in two dosing regimens alone and in combination with anti-PD-1

FIG. 4A: Mouse CSF-1 levels in mouse plasma after treatment of BLZ945 alone and in combination with anti-PD-1 at two different schedules

FIG. 4B: Mouse CSF-1 levels in mouse plasma after treatment of BLZ945 alone and in combination with anti-PD-1 at two different schedules

FIG. 5A: Immunomodulation of TAM and Tregs after treatment with BLZ945 for 4 days on/3 days off (mouse model)

FIG. 5B (FIG. 4-8B): Immunomodulation of TAM and Tregs aftertreatment with BLZ945 for 4 days on/3 days off (mouse model)

FIG. 6: AST Elevation over Single Agent Dose Groups (clinical data)

FIG. 7: ALT Elevation over Single Agent Dose Groups (clinical data)

FIG. 8: Analysis of microglial depletion following consecutive dosing days of ALS mice models with BLZ945

FIG. 9: Analysis of microglial reduction with several doses of BLZ945

FIG. 10: Microglial depletion in ALS SOD1^{G93A} mice with b.i.d. dosing

FIG. 11: Microglial depletion in ALS SOD1^{G93A} mice in the efficacy study

FIG. 12: Dose-dependent effect of BLZ945 on body weight accumulation

FIG. 13: Effect of BLZ945 on grip strength and CMAP

### DETAILED DESCRIPTION

The invention can be practiced with an inhibitor of CSF-1R capable of penetrating the brain. Some such compounds are the 6-O-substituted benzoxazole and benzothiazole compounds disclosed in WO2007/121484, particularly the compounds of Formula IIa and IIb in that reference, and the compounds disclosed herein.

In one aspect, the invention provides a CSF-1R inhibitor of Formula (I) 4-((2-(((1R,2R)-2-hydroxycyclohexyl)amino)benzo[d]thiazol-6-yl)oxy)-N-methylpicolinamide:

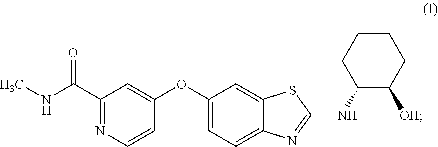 [SMILES: CNC(=O)c1cc(Oc2ccc3nc(N[C@@H]4CCCC[C@H]4O)sc3c2)ccn1]

or a pharmaceutically acceptable salt thereof, for use in the treatment of cancer, wherein (I) is administered 4 days-on and 10 days-off, twice per cycle.

In another aspect, the invention provides the CSF-1R compound for use in the treatment of cancer, wherein each cycle is 28 days.

In an additional aspect, the invention provides the CSF-1R compound for use in the treatment of cancer, wherein the compound of Formula (I) is administered twice daily.

In another aspect, the invention provides the CSF-1R compound for use in the treatment of cancer, wherein the daily dose of the compound of Formula (I) is 100 mg/day, 150 mg/day, 200 mg/day, 300 mg/day, 400 mg/day, 500 mg/day, 600 mg/day, 700 mg/day, 900 mg/day, or 1200 mg/day.

In a further aspect, the invention provides the CSF-1R compound for use in the treatment of cancer, wherein the daily dose is 700 mg/day.

In another aspect, the invention provides the CSF-1R compound for use in the treatment of cancer, wherein the cancer is leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastoma, or melanoma.

In a further aspect, the invention provides the CSF-1R compound for use in the treatment of cancer, wherein the cancer is glioblastoma.

In one aspect, the invention provides a pharmaceutical combination comprising (i) a CSF-1R inhibitor of Formula (I) 4-((2-(((1R,2R)-2-hydroxycyclohexyl)amino)benzo[d]thiazol-6-yl)oxy)-N-methylpicolinamide, or a pharmaceutically acceptable salt thereof, and (ii) anti-PD-1 antibody molecule or a pharmaceutically acceptable salt thereof, for use in the treatment of cancer, wherein (i) is administered 4 days-on and 10 days-off, twice per cycle and (ii) is administered at least once per cycle.

In an additional aspect, the invention provides the pharmaceutical combination for use in the treatment of cancer, wherein the CSF-1R inhibitor of Formula (I) is only administered on 3 of the 4 days-on.

In a further aspect, the invention provides the pharmaceutical combination for use in the treatment of cancer, wherein each cycle is 28 days.

In another aspect, the invention provides the pharmaceutical combination for use in the treatment of cancer, wherein (i) is administered twice daily.

In another aspect, the invention provides, the pharmaceutical combination for use in the treatment of cancer, wherein the daily dose of (i) is 100 mg/day, 150 mg/day, 200 mg/day, 300 mg/day, 400 mg/day, 500 mg/day, 600 mg/day, 700 mg/day, 900 mg/day, or 1200 mg/day.

In a further aspect, the invention provides the pharmaceutical combination for use in the treatment of cancer, wherein the daily dose of (i) is 1200 mg/day.

In another aspect, the invention provides the pharmaceutical combination for use in the treatment of cancer, wherein (ii) is administered, every 4 weeks.

In an additional aspect, the invention provides the pharmaceutical combination for use in the treatment of cancer, wherein (ii) is selected from nivolumab, pembrolizumab, pidilizumab, spartalizumab, or a pharmaceutical salt thereof.

In a further aspect, the invention provides the pharmaceutical combination for use in the treatment of cancer, wherein (ii) is spartalizumab, or a pharmaceutical salt thereof.

In an additional aspect, the invention provides the pharmaceutical combination for use in the treatment of cancer, wherein (ii) is administered intravenously in a single dose of 300 to 400 mg/day.

In a further aspect, the invention provides the pharmaceutical combination for use in the treatment of cancer, wherein the single dose of (ii) is 400 mg/day.

In another aspect, the invention provides the pharmaceutical combination for use in the treatment of cancer, wherein the cancer is leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastoma, or melanoma.

In a further aspect, the invention provides the pharmaceutical combination for use in the treatment of cancer, wherein the cancer is glioblastoma.

The CSF-1R inhibitor to be combined with the anti-PD-1 antibody molecule, or a pharmaceutical salt thereof, is 4-((2-(((1R,2R)-2-hydroxycyclohexyl)amino)benzo[d]thiazol-6-yl)oxy)-N-methylpicolinamide, or a pharmaceutically acceptable salt thereof, of formula (I)

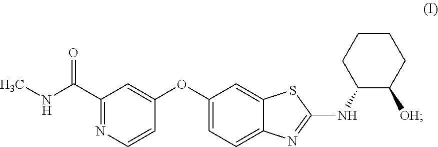 [SMILES: CNC(=O)c1cc(Oc2ccc3nc(N[C@@H]4CCCC[C@H]4O)sc3c2)ccn1]

as disclosed in WO2007/121484 (Example 157).

Therefore the present disclosure also provides a pharmaceutical combination comprising (i) 4-((2-(((1R,2R)-2-hydroxycyclohexyl)amino)benzo[d]thiazol-6-yl)oxy)-N-methylpicolinamide, or a pharmaceutically acceptable salt thereof, and (ii) an anti-PD-1 antibody molecule or a pharmaceutically acceptable salt thereof, for use in the treatment of cancer, wherein the compound of Formula (I), namely 4-((2-(((1R,2R)-2-hydroxycyclohexyl)amino)benzo[d]thiazol-6-yl)oxy)-N-methylpicolinamide, or a pharmaceutically acceptable salt thereof, is administered 4 days-on and 10 days-off, twice per cycle and anti-PD-1 antibody molecule (ii) as described herein is administered at least once per cycle.

In another aspect the invention provides a CSF-1R inhibitor of Formula (I), 4-((2-(((1R,2R)-2-hydroxycyclohexyl)amino)benzo[d]thiazol-6-yl)oxy)-N-methylpicolinamide:

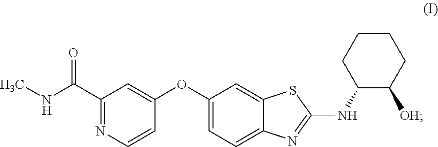 [SMILES: CNC(=O)c1cc(Oc2ccc3nc(N[C@@H]4CCCC[C@H]4O)sc3c2)ccn1]

or a pharmaceutically acceptable salt thereof, for use in the treatment of an neuro-inflammatory disease modulated by CSF-1R, wherein the CSF-1R inhibitor is administered during an on period, followed by an off period wherein the CSF-1R inhibitor is not administered, and wherein at least one pair of on period and off period makes up a cycle.

In a further aspect, the invention provides a CSF-1R inhibitor of Formula (I) or a pharmaceutically acceptable salt thereof for use in the treatment of a neuro-inflammatory disease, wherein the CSF-1R inhibitor is administered 4 days on.

In an additional aspect, the invention provides a CSF-1R inhibitor of Formula (I) or a pharmaceutically acceptable salt thereof for use in the treatment of a neuro-inflammatory disease, wherein the CSF-13R inhibitor is administered 7 days on.

In a further aspect, the invention provides a CSF-1R inhibitor of Formula (J) or a pharmaceutically acceptable salt thereof for use in the treatment of a neuro-inflammatory disease, wherein the CSF-1R inhibitor is administered 4 days on followed by up to 10 days-off, preferably between 7 to 10 days off, more preferably 10 days off.

In a further aspect, the invention provides a CSF-1R inhibitor of Formula (I) or a pharmaceutically acceptable salt thereof for use in the treatment of a neuro-inflammatory disease, wherein the CSF-1R inhibitor is administered 4 days on and 10 days-off.

In a further aspect, the invention provides a CSF-1R inhibitor of Formula (I) or a pharmaceutically acceptable salt thereof for use in the treatment of a neuro-inflammatory disease, wherein the CSF-1R inhibitor is administered 7 days on and 7 days-off.

In a further aspect, the invention provides a CSF-1R inhibitor of Formula (I) or a pharmaceutically acceptable salt thereof for use in the treatment of a neuro-inflammatory disease, wherein the CSF-1R inhibitor is administered twice daily.

In a further aspect, the invention provides a CSF-1R inhibitor of Formula (I) or a pharmaceutically acceptable salt thereof for use in the treatment of a neuro-inflammatory disease, wherein the daily dose of the CSF-1R inhibitor of Formula (I) is 100 mg/day, 150 mg/day, 200 mg/day, 300 mg/day, 400 mg/day, 500 mg/day, 600 mg/day, 700 mg/day, 900 mg/day, or 1200 mg/day.

In a further aspect, the invention provides a CSF-1R inhibitor of Formula (I) or a pharmaceutically acceptable salt thereof for use in the treatment of a neuro-inflammatory disease, wherein the daily dose is 300 mg, 600 mg, or 1200 mg/day, preferably 1200 mg.

In a further aspect, the invention provides a CSF-1R inhibitor of Formula (I) or a pharmaceutically acceptable salt thereof for use in the treatment of a neuro-inflammatory disease, wherein the neuro-inflammatory disease is amyotrophic lateral sclerosis.

In a further aspect, the invention provides a pharmaceutical combination comprising (i) a CSF-1R inhibitor of Formula (I) 4-((2-(((1R,2R)-2-hydroxycyclohexyl)amino)benzo[d]thiazol-6-yl)oxy)-N-methylpicolinamide, or a pharmaceutically acceptable salt thereof, and (ii) any one of: edaravone, or riluzole; for use in the treatment of a neuro-inflammatory disease.

Enumerated embodiments relating to the use of a CSF-1R inhibitor in the treatment of cancer:

Embodiment 1.1: A CSF-1R inhibitor of Formula (I) 4-((2-(((1R,2R)-2-hydroxycyclohexyl)amino)benzo[d]thiazol-6-yl)oxy)-N-methylpicolinamide:

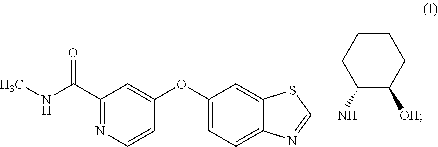 [SMILES: CNC(=O)c1cc(Oc2ccc3nc(N[C@@H]4CCCC[C@H]4O)sc3c2)ccn1]

or a pharmaceutically acceptable salt thereof, for use in the treatment of cancer, wherein (I) is administered 4 days-on and 10 days-off, twice per cycle.

Embodiment 1.2: The CSF-1R compound of embodiment 1.1 for use in the treatment of cancer, wherein each cycle is 28 days.
Embodiment 1.3: The CSF-1R compound of embodiment 1.1 for use in the treatment of cancer, wherein the compound of Formula (I) is administered twice daily.
Embodiment 1.4: The CSF-1R compound of any one of embodiments 1.1 to 1.3 for use in the treatment of cancer, wherein the daily dose of the compound of Formula (I) is 100 mg/day, 150 mg/day, 200 mg/day, 300 mg/day, 400 mg/day, 500 mg/day, 600 mg/day, 700 mg/day, 900 mg/day, or 1200 mg/day.
Embodiment 1.5: The CSF-1R compound of embodiment 1.4, wherein the daily dose is 700 mg/day.
Embodiment 1.6: The CSF-1R compound of any one of embodiments 1.1 to 1.5 for use in the treatment of cancer, wherein the cancer is leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastoma, or melanoma.
Embodiment 1.7: The CSF-1R compound of embodiment 1.6 for use in the treatment of cancer, wherein the cancer is glioblastoma.
Embodiment 1.8: A pharmaceutical combination comprising (i) a CSF-1R inhibitor of Formula (I) 4-((2-(((1R,2R)-2-hydroxycyclohexyl)amino)benzo[d]thiazol-6-yl)oxy)-N-methylpicolinamide, or a pharmaceutically acceptable salt thereof, and (ii) anti-PD-1 antibody molecule or a pharmaceutically acceptable salt thereof, for use in the treatment of cancer, wherein (i) is administered 4 days-on and 10 days-off, twice per cycle and (ii) is administered at least once per cycle.
Embodiment 1.9: The pharmaceutical combination for use in the treatment of cancer according to embodiment 1.8, wherein each cycle is 28 days.
Embodiment 1.10: The pharmaceutical combination for use in the treatment of cancer according to embodiment 1.8, wherein (i) is administered twice daily.
Embodiment 1.11: The pharmaceutical combination for use in the treatment of cancer according to any one of embodiments 1.8 to 1.10, wherein the daily dose of (i) is 100 mg/day, 150 mg/day, 200 mg/day, 300 mg/day, 400 mg/day, 500 mg/day, 600 mg/day. 700 mg/day, 900 mg/day, or 1200 mg/day.
Embodiment 1.12: The pharmaceutical combination for use in the treatment of cancer according to embodiment 1.11, wherein the daily dose of (i) is 1200 mg/day.
Embodiment 1.13: The pharmaceutical combination for use in the treatment of cancer according to embodiment 1.8, wherein (ii) is administered, every 4 weeks.
Embodiment 1.14: The pharmaceutical combination for use in the treatment of cancer according to any one of embodiments 1.8 to 1.13, wherein (ii) is selected from nivolumab, pembrolizumab, pidilizumab, spartalizumab, or a pharmaceutical salt thereof.
Embodiment 1.15: The pharmaceutical combination for use in the treatment of cancer according to embodiment 1.14, wherein (ii) is spartalizumab, or a pharmaceutical salt thereof.
Embodiment 1.16: The pharmaceutical combination for use in the treatment of cancer according to any one of embodiments 1.8 to 1.15, wherein (ii) is administered intravenously in a single dose of 300 to 400 mg/day.
Embodiment 1.17: The pharmaceutical combination for use in the treatment of cancer according to embodiment 1.16, wherein the single dose is 400 mg/day.
Embodiment 1.18: The pharmaceutical combination for use in the treatment of cancer according to embodiments 1.8 to 1.17, wherein the cancer is leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastoma, or melanoma.
Embodiment 1.19: The pharmaceutical combination of embodiment 1.18 for use in the treatment of cancer, wherein the cancer is glioblastoma.
Embodiment 1.20: A method for treating cancer in a subject in need thereof, comprising administering a CSF-1R inhibitor of Formula (I) 4-((2-(((1R,2R)-2-hydroxycyclohexyl)amino)benzo[d]thiazol-6-yl)oxy)-N-methylpicolinamide:

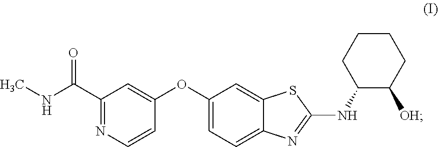 [SMILES: CNC(=O)c1cc(Oc2ccc3nc(N[C@@H]4CCCC[C@H]4O)sc3c2)ccn1]

or a pharmaceutically acceptable salt thereof, wherein (I) is administered 4 days-on and 10 days-off, twice per cycle.

Embodiment 1.21: The method of embodiment 1.20, wherein each cycle is 28 days.
Embodiment 1.22: The method of embodiment 1.20, wherein the compound of Formula (I) is administered twice daily.
Embodiment 1.23: The method of any one of embodiments 1.20 to 1.22 for use in the treatment of cancer, wherein the daily dose of the compound of Formula (I) is 100 mg/day, 150 mg/day, 200 mg/day, 300 mg/day, 400 mg/day, 500 mg/day. 600 mg/day, 700 mg/day. 900 mg/day, or 1200 mg/day.
Embodiment 1.24: The method of embodiment 1.23, wherein the daily dose is 700 mg/day.
Embodiment 1.25: The method of any one of embodiments 1.20 to 1.24, wherein the cancer is leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastoma, or melanoma.
Embodiment 1.26: The method of embodiment 1.25, wherein the cancer is glioblastoma.

Enumerated embodiments relating to the use of a CSF-1R inhibitor in the treatment of neuro-inflammatory diseases:

Embodiment 2.1: A CSF-1R inhibitor of Formula (I), 4-((2-(((1R,2R)-2-hydroxycyclohexyl)amino)benzo[d]thiazol-6-yl)oxy)-N-methylpicolinamide:

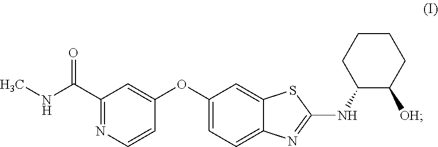 [SMILES: CNC(=O)c1cc(Oc2ccc3nc(N[C@@H]4CCCC[C@H]4O)sc3c2)ccn1]

or a pharmaceutically acceptable salt thereof, for use in the treatment of an neuro-inflammatory disease modulated by CSF-1R, wherein the CSF-1R inhibitor is administered during an on period, followed by an off period wherein the CSF-1R inhibitor is not administered, and wherein at least one pair of on period and off period makes up a cycle.
Embodiment 2.2: The CSF-1R inhibitor of Formula (I) or a pharmaceutically acceptable salt thereof for use in the treatment of a disease according to embodiment 2.1, wherein the CSF-1R inhibitor is administered 4 days on.
Embodiment 2.3: The CSF-1R inhibitor of Formula (I) or a pharmaceutically acceptable salt thereof for use in the treatment of a disease according to embodiment 2.1, wherein the CSF-1R inhibitor is administered 7 days on.
Embodiment 2.4: The CSF-1R inhibitor of Formula (I) or a pharmaceutically acceptable salt thereof for use in the treatment of a disease according to embodiment 2.1, wherein the CSF-1R inhibitor is administered 4 days on followed by up to 10 days-off, preferably between 7 to 10 days off, more preferably 10 days off.
Embodiment 2.5: The CSF-1R inhibitor of Formula (I) or a pharmaceutically acceptable salt thereof for use in the treatment of a disease according to embodiment 2.1, wherein the CSF-1R inhibitor is administered 4 days on and 10 days-off.
Embodiment 2.6: The CSF-1R inhibitor of Formula (I) or a pharmaceutically acceptable salt thereof for use in the treatment of a disease according to embodiment 2.1, wherein the CSF-1R inhibitor is administered 7 days on and 7 days-off.
Embodiment 2.7: The CSF-1R inhibitor of Formula (I) or a pharmaceutically acceptable salt thereof for use according to any one of embodiments 2.1 to 2.6, wherein the CSF-1R inhibitor is administered twice daily.
Embodiment 2.8: The CSF-1R inhibitor of Formula (I) or a pharmaceutically acceptable salt thereof for use according to any one of embodiments 2.1 to 2.7, wherein the daily dose of the CSF-1R inhibitor of Formula (I) is 100 mg/day. 150 mg/day, 200 mg/day, 300 mg/day. 400 mg/day, 500 mg/day, 600 mg/day, 700 mg/day, 900 mg/day, or 1200 mg/day.
Embodiment 2.9: The CSF-1R inhibitor of Formula (I) or a pharmaceutically acceptable salt thereof for use according to embodiment 2.8, wherein the daily dose is 300 mg, 600 mg, or 1200 mg/day, preferably 1200 mg.
Embodiment 2.10: The CSF-1R inhibitor of Formula (I) or a pharmaceutically acceptable salt thereof for use according to any one of embodiments 2.1 to 2.9, wherein the neuro-inflammatory disease is Alzheimer's disease, multiple sclerosis, or amyotrophic lateral sclerosis.
Embodiment 2.11: The CSF-1R inhibitor of Formula (I) or a pharmaceutically acceptable salt thereof for use according to any one of embodiments 2.1 to 2.9, wherein the neuro-inflammatory disease is amyotrophic lateral sclerosis.
Embodiment 2.12: A pharmaceutical combination comprising (i) a CSF-1R inhibitor of Formula (I) 4-((2-(((1R,2R)-2-hydroxycyclohexyl)amino)benzo[d]thiazol-6-yl)oxy)-N-methylpicolinamide, or a pharmaceutically acceptable salt thereof, and (ii) any one of: edaravone, or riluzole; for use in the treatment of according to any one of embodiments 2.1 to 2.10.
Embodiment 2.13: A method for treating a neuro-inflammatory disease modulated by CSF-1R in a subject in need thereof, comprising a administering a CSF-1R inhibitor of Formula (I), 4-((2-(((1R,2R)-2-hydroxycyclohexyl)amino)benzo[d]thiazol-6-yl)oxy)-N-methylpicolinamide:

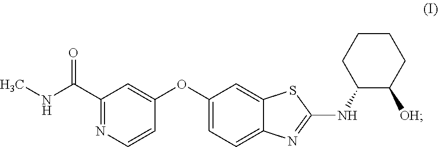 [SMILES: CNC(=O)c1cc(Oc2ccc3nc(N[C@@H]4CCCC[C@H]4O)sc3c2)ccn1]

or a pharmaceutically acceptable salt thereof, wherein the CSF-1R inhibitor is administered during an on period, followed by an off period wherein the CSF-1R inhibitor is not administered, and wherein at least one pair of on period and off period makes up a cycle.
Embodiment 2.14: The method of embodiment 2.13, wherein the CSF-1R inhibitor is administered 4 days on.
Embodiment 2.15: The method of embodiment 2.14, wherein the CSF-1R inhibitor is administered 7 days on.
Embodiment 2.16: The method of embodiment 2.13, wherein the CSF-1R inhibitor is administered 4 days on followed by up to 10 days-off, preferably between 7 to 10 days off, more preferably 10 days off.
Embodiment 2.17: The method of embodiment 2.13, wherein the CSF-1R inhibitor is administered 4 days on and 10 days-off.
Embodiment 2.18: The method of embodiment 2.13, wherein the CSF-1R inhibitor is administered 7 days on and 7 days-off.
Embodiment 2.19: The method according to any one of embodiments 2.13 to 2.18, wherein the CSF-1R inhibitor is administered twice daily.
Embodiment 2.20: The method according to any one of embodiments 2.13 to 2.19, wherein the daily dose of the CSF-1R inhibitor of Formula (I) is 100 mg/day, 150 mg/day, 200 mg/day, 300 mg/day, 400 mg/day. 500 mg/day, 600 mg/day, 700 mg/day, 900 mg/day, or 1200 mg/day.
Embodiment 2.21: The method of embodiment 2.20, wherein the daily dose is 300 mg/day, 600 mg/day, or 1200 mg/day, preferably 1200 mg/day.
Embodiment 2.22: The method of according to any one of embodiments 2.13 to 2.21, wherein the neuro-inflammatory disease is amyotrophic lateral sclerosis.

In the present disclosure the term “pharmaceutical combination” refers to a non-fixed combination. The term “non-fixed combination” means that the active ingredients, e.g. compound of Formula (I), namely 4-((2-(((1R,2R)-2-hydroxycyclohexyl)amino)benzo[d]thiazol-6-yl)oxy)-N-methylpicolinamide, or a pharmaceutically acceptable salt thereof and an anti-PD-1 antibody molecule, or a pharmaceutically acceptable salt form, are both administered to a patient as separate entities either simultaneously or sequentially with no specific time limits, wherein such administration provides therapeutically effective levels of the two compounds in the body of the patient.

The terms “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

The anti-PD-1 antibody molecule, or a pharmaceutically acceptable salts thereof, that can be used in combination with CSF-1R inhibitors of the present disclosure, is any anti-PD-1 antibody as disclosed herein. For example, the anti-PD-1 antibody molecule can comprise at least one antigen-binding region. e.g., a variable region or an antigen-binding fragment thereof, from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD1 antibody molecule is preferably selected from nivolumab (Opdivo), pembrolizumab (Keytruda), pidilizumab, spartalizumab, or a pharmaceutical salt thereof. Most preferably, the anti-PD-1 antibody molecule is spartalizumab, or a pharmaceutical salt thereof. The anti-PD-1 antibody molecule designated as spartalizumab was described in PCT/CN2016/099494. More particularly the PDR-001 inhibitor, or a pharmaceutically acceptable salt thereof, comprises a heavy chain variable region (VH) comprising a HCDR1, a HCDR2 and a HCDR3 amino acid sequence of BAP049-Clone-E and a light chain variable region (VL) comprising a LCDR1, a LCDR2 and a LCDR3 amino acid sequence of BAP049-Clone-E as described in Table 1.

The anti-PD-1 antibody molecule, or a pharmaceutically acceptable salt thereof, of the present disclosure comprises, for example, at least one, two, three or four variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure comprises, for example, at least one or two heavy chain variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure comprises, for example, at least one or two light chain variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure includes, for example, a heavy chain constant region for an IgG4, e.g., a human IgG4. The human IgG4 includes a substitution at position 228 according to EU numbering (e.g., a Ser to Pro substitution). The anti-PD-1 antibody molecule includes a heavy chain constant region for an IgG1, e.g., a human IgG1. The human IgG1 includes a substitution at position 297 according to EU numbering (e.g., an Asn to Ala substitution). The human IgG1 may also include a substitution at position 265 according to EU numbering, a substitution at position 329 according to EU numbering, or both (e.g., an Asp to Ala substitution at position 265 and/or a Pro to Ala substitution at position 329). The human IgG1 also includes a substitution at position 234 according to EU numbering, a substitution at position 235 according to EU numbering, or both (e.g., a Leu to Ala substitution at position 234 and/or a Leu to Ala substitution at position 235). The heavy chain constant region comprises an amino sequence set forth in Table 3, or a sequence substantially identical (e.g., at least 80%, 85%, 90%, 92%, 95%, 97%, 98%, 99% or higher identical) thereto.

The anti-PD-1 antibody molecule, or a pharmaceutically acceptable salt thereof, according to the present disclosure includes, for example, a kappa light chain constant region, e.g., a human kappa light chain constant region. The light chain constant region comprises an amino sequence set forth in Table 3, or a sequence substantially identical (e.g., at least 80%, 85%, 90%, 92%, 95%, 97%, 98%, 99% or higher identical) thereto.

The anti-PD-1 antibody molecule, or a pharmaceutically acceptable salt thereof, according to the present disclosure also includes, for example, a heavy chain constant region for an IgG4, e.g., a human IgG4, and a kappa light chain constant region, e.g., a human kappa light chain constant region, e.g., a heavy and light chain constant region comprising an amino sequence set forth in Table 3, or a sequence substantially identical (e.g., at least 80%, 85%, 90%, 92%, 95%, 97%, 98%, 99% or higher identical) thereto. The human IgG4 includes a substitution at position 228 according to EU numbering (e.g., a Ser to Pro substitution). The anti-PD-1 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 3, or a sequence substantially identical (e.g., at least 80%, 85%, 90%, 92%, 95%, 97%, 98%, 99% or higher identical) thereto. The human IgG1 may also include a substitution at position 297 according to EU numbering (e.g., an Asn to Ala substitution). The human IgG1 includes a substitution at position 265 according to EU numbering, a substitution at position 329 according to EU numbering, or both (e.g., an Asp to Ala substitution at position 265 and/or a Pro to Ala substitution at position 329). The human IgG1 includes a substitution at position 234 according to EU numbering, a substitution at position 235 according to EU numbering, or both (e.g., a Leu to Ala substitution at position 234 and/or a Leu to Ala substitution at position 235).

The anti-PD-1 antibody molecule, or a pharmaceutically acceptable salt thereof, of the present disclosure also includes, for example, a heavy chain variable domain and a constant region, a light chain variable domain and a constant region, or both, comprising the amino acid sequence of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as shown in Table 4; or a sequence substantially identical thereto.

The anti-PD-1 antibody molecule, or a pharmaceutically acceptable salt thereof, according to the present disclosure includes at least one, two, or three complementary determining regions (CDRs) from a heavy chain variable region of an antibody described herein. e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, according to the present disclosure includes, for example, at least one, two, or three CDRs (or collectively all of the CDRs) from a heavy chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, according to the present disclosure includes, for example, at least one, two, or three CDRs from a light chain variable region of an antibody described herein, e.g., an antibody chosen from any of BAP049-Clone-6 or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequence.

The anti-PD-1 antibody molecule, or a pharmaceutically acceptable salt thereof, includes, for example, at least one, two, or three CDRs (or collectively all of the CDRs) from a heavy chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, includes, for example, at least one, two, or three CDRs from a light chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequence.

The anti-PD-1 antibody molecule, or a pharmaceutically acceptable salt thereof, includes, for example, at least one, two, or three CDRs (or collectively all of the CDRs) from a light chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1. In certain embodiments, the anti-PD-1 antibody molecule, or a pharmaceutically acceptable salt thereof, includes a substitution in a light chain CDR, e.g., one or more substitutions in a CDR1, CDR2 and/or CDR3 of the light chain. The anti-PD-1 antibody molecule, or a pharmaceutically acceptable salt thereof, includes a substitution in the light chain CDR3 at position 102 of the light variable region, e.g., a substitution of a cysteine to tyrosine, or a cysteine to serine residue, at position 102 of the light variable region according to Table 1 (e.g., SEQ ID NO: 54 or 70 for a modified sequence).

The anti-PD-1 antibody molecule, or a pharmaceutically acceptable salt thereof, according to the present disclosure includes, for example, at least one, two, three, four, five or six CDRs (or collectively all of the CDRs) from a heavy and light chain variable region comprising an amino acid sequence shown in Table 1, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, of the present disclosure includes, for example, all six CDRs from an antibody described herein, e.g., an antibody chosen from any of BAP049-Clone-B or BAP049-Clone-E; or as described in Table 1, or encoded by the nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). The anti-PD-1 antibody molecule, or a pharmaceutically acceptable salt thereof, may also include any CDR described herein. The anti-PD-1 antibody molecule, or a pharmaceutically acceptable salt thereof, includes a substitution in a light chain CDR, e.g., one or more substitutions in a CDR1, CDR2 and/or CDR3 of the light chain. The anti-PD-1 antibody molecule, or a pharmaceutically acceptable salt thereof, according to the present disclosure, includes a substitution in the light chain CDR3 at position 102 of the light variable region, e.g., a substitution of a cysteine to tyrosine, or a cysteine to serine residue, at position 102 of the light variable region according to Table 1.

The anti-PD-1 antibody molecule, or a pharmaceutically acceptable salt thereof, of the present disclosure, includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al, shown in Table 1.

The anti-PD-1 antibody molecule, or a pharmaceutically acceptable salt thereof, of the present invention, includes, for example, at least one, two, or three CDRs according to Kabat et al. (e.g., at least one, two, or three CDRs according to the Kabat definition as set out in Table 1) from a light chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

The anti-PD-1 antibody molecule, or a pharmaceutically acceptable salt thereof, according to the present disclosure, includes, for example, at least one, two, three, four, five, or six CDRs according to Kabat et a. (e.g., at least one, two, three, four, five, or six CDRs according to the Kabat definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%. 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

The anti-PD-1 antibody molecule, or a pharmaceutically acceptable salt thereof, of the present disclosure, includes all six CDRs according to Kabat et al. (e.g., all six CDRs according to the Kabat definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. The anti-PD-1 antibody molecule, or a pharmaceutically acceptable salt thereof, according to the present disclosure, may include any CDR described herein.

The anti-PD-1 antibody molecule, or a pharmaceutically acceptable salt thereof, according to the present disclosure, includes, for example at least one, two, or three Chothia hypervariable loops (e.g., at least one, two, or three hypervariable loops according to the Chothia definition as set out in Table 1) from a heavy chain variable region of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

The anti-PD-1 antibody molecule, or a pharmaceutically acceptable salt thereof, according to the present disclosure, includes, for example, at least one, two, or three Chothia hypervariable loops (e.g., at least one, two, or three hypervariable loops according to the Chothia definition as set out in Table 1) of a light chain variable region of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

The anti-PD-1 antibody molecule, or a pharmaceutically acceptable salt thereof, according to the present disclosure, includes, for example, at least one, two, three, four, five, or six hypervariable loops (e.g., at least one, two, three, four, five, or six hypervariable loops according to the Chothia definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 1.

The anti-PD-1 antibody molecule, or a pharmaceutically acceptable salt thereof, according to the present disclosure, includes, for example, all six hypervariable loops (e.g., all six hypervariable loops according to the Chothia definition as set out in Table 1) of an antibody described herein, e.g., an antibody chosen from any of BAP049-Clone-B or BAP049-Clone-E, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 1. The anti-PD-1 antibody molecule, or a pharmaceutically acceptable salt thereof, according to the present disclosure may include any hypervariable loop described herein.

The anti-PD-1 antibody molecule, or a pharmaceutically acceptable salt thereof, according to the present disclosure, includes, for example, at least one, two, or three hypervariable loops that have the same canonical structures as the corresponding hypervariable loop of an antibody described herein, e.g., an antibody chosen from any of BAP049-Clone-B or BAP049-Clone-E, e.g., the same canonical structures as at least loop 1 and/or loop 2 of the heavy and/or light chain variable domains of an antibody described herein. (See, e.g., Chothia et al. J. Mol. Biol. 1992, 227, 799; Tomlinson et al. J. Mol. Biol. 1992, 227:776-798 for descriptions of hypervariable loop canonical structures). These structures can be determined by inspection of the tables described in these references.

The anti-PD-1 antibody molecule, or a pharmaceutically acceptable salt thereof, according to the present disclosure, may also include, for example, a combination of CDRs or hypervariable loops defined according to the Kabat et al. and Chothia et al. as described herein in Table 1.

The anti-PD-1 antibody molecule, or a pharmaceutically acceptable salt thereof, according to the present disclosure, includes, for example, at least one, two or three CDRs or hypervariable loops from a heavy chain variable region of an antibody described herein, e.g., an antibody chosen from any of BAP049-Clone-B or BAP049-Clone-E, according to the Kabat and Chothia definition (e.g., at least one, two, or three CDRs or hypervariable loops according to the Kabat and Chothia definition as set out in Table 1); or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 1.

For example, the anti-PD-1 antibody molecule, or a pharmaceutically acceptable salt thereof, according to the present disclosure, can include VH CDR1 according to Kabat et al. or VH hypervariable loop 1 according to Chothia et al., or a combination thereof, e.g., as shown in Table 1. The combination of Kabat and Chothia CDR of VH CDR1 comprises the amino acid sequence GYTFTTYWMH (SEQ ID NO: 224), or an amino acid sequence substantially identical thereto (e.g., having at least one amino acid alteration, but not more than two, three or four alterations (e.g., substitutions, deletions, or insertions, e.g., conservative substitutions). The anti-PD-1 antibody molecule can further include, e.g., VH CDRs 2-3 according to Kabat et al. and VL CDRs 1-3 according to Kabat et al., e.g., as shown in Table 1. Accordingly, the framework regions (FW) are defined based on a combination of CDRs defined according to Kabat et al. and hypervariable loops defined according to Chothia et al. For example, the anti-PD-1 antibody molecule can include VH FW1 defined based on VH hypervariable loop 1 according to Chothia et al. and VH FW2 defined based on VH CDRs 1-2 according to Kabat et al., e.g., as shown in Table 1. The anti-PD-1 antibody molecule can further include, e.g., VH FWs 3-4 defined based on VH CDRs 2-3 according to Kabat et al. and VL FWs 1-4 defined based on VL CDRs 1-3 according to Kabat et aW.

The anti-PD-1 antibody molecule, or a pharmaceutically acceptable salt thereof, according to the present disclosure, includes at least one, two or three CDRs from a light chain variable region of an antibody described herein, e.g., an antibody chosen from any of BAP049-Clone-B or BAP049-Clone-E, according to the Kabat and Chothia definitions (e.g., at least one, two, or three CDRs according to the Kabat and Chothia definitions as set out in Table 1).

The anti-PD-1 antibody molecule, or a pharmaceutically acceptable salt thereof, according to the present disclosure, includes:

(a) a heavy chain variable region (VH) comprising a VHCDR1 amino acid sequence of SEQ ID NO: 4, a VHCDR2 amino acid sequence of SEQ ID NO: 5, and a VHCDR3 amino acid sequence of SEQ ID NO: 3; and a light chain variable region (VL) comprising a VLCDR1 amino acid sequence of SEQ ID NO: 13, a VLCDR2 amino acid sequence of SEQ ID NO: 14, and a VLCDR3 amino acid sequence of SEO ID NO: 33;
(b) a VH comprising a VHCDR1 amino acid sequence chosen from SEQ ID NO: 1; a VHCDR2 amino acid sequence of SEQ ID NO: 2: and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32;
(c) a VH comprising a VHCDR1 amino acid sequence of SEQ ID NO: 224, a VHCDR2 amino acid sequence of SEQ ID NO: 5, and a VHCDR3 amino acid sequence of SEQ ID NO: 3; and a VL comprising a VLCDR1 amino acid sequence of SEQ ID NO: 13, a VLCDR2 amino acid sequence of SEQ ID NO: 14, and a VLCDR3 amino acid sequence of SEQ ID NO: 33; or
(d) a VH comprising a VHCDR1 amino acid sequence of SEQ ID NO: 224; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32.

The anti-PD-1 antibody molecule, or a pharmaceutically acceptable salt thereof, according to the present disclosure, comprises a heavy chain variable region (VH) comprising a VHCDR1 amino acid sequence chosen from SEQ ID NO: 1, SEQ ID NO: 4, or SEQ ID NO: 224; a VHCDR2 amino acid sequence of SEQ ID NO: 2 or SEQ ID NO: 5; and a VHCDR3 amino acid sequence of SEQ ID NO: 3; and a light chain variable region (VL) comprising a VLCDR1 amino acid sequence of SEQ ID NO: 10 or SEQ ID NO: 13, a VLCDR2 amino acid sequence of SEQ ID NO: 11 or SEQ ID NO: 14, and a VLCDR3 amino acid sequence of SEQ ID NO: 32 or SEQ ID NO: 33.

The anti-PD-1 antibody molecule, or a pharmaceutically acceptable salt thereof, according to the present disclosure, can comprise, for example, a heavy chain variable domain comprising the amino acid sequence of SEQ ID NO: 38 and a light-chain variable domain comprising the amino acid sequence of SEQ ID NO: 70.

The anti-PD-1 antibody molecule, or a pharmaceutically acceptable salt thereof, according to the present disclosure, can comprise, for example, a heavy chain comprising the amino acid sequence of SEQ ID NO: 91 and a light chain comprising the amino acid sequence of SEQ ID NO: 72.

The anti-PD-1 antibody molecule, or a pharmaceutically acceptable salt thereof, according to the present disclosure, comprises a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1.

The anti-PD-1 antibody molecule, or a pharmaceutically acceptable salt thereof, according to the present disclosure, comprises a heavy chain variable region (VH) comprising a HCDR1, a HCDR2 and a HCDR3 amino acid sequence of BAP049-Clone-E as described in Table 1 and a light chain variable region (VL) comprising a LCDR1, a LCDR2 and a LCDR3 amino acid sequence of BAP049-Clone-E as described in Table 1.

It is understood that the anti-PD-1 antibody molecule, or the anti-PD-1 antibody molecule, of the present disclosure may have additional conservative or non-essential amino acid substitutions, which do not have a substantial effect on their functions.

The term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). An antibody molecule comprises a full length antibody, or a full length immunoglobulin chain, or an antigen binding or functional fragment of a full length antibody, or a full length immunoglobulin chain. An antibody molecule can also be a multi-specific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope.

The term “Pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compound herein is to be understood as referring also to the corresponding salt, as appropriate and expedient. The salts of the inhibitors, as described herein, are preferably pharmaceutically acceptable salts; suitable counter-ions forming pharmaceutically acceptable salts are known in the field.

The term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

The term “inhibition” or “inhibitor” includes a reduction in a certain parameter, e.g., an activity, of a given molecule, e.g., an immune checkpoint inhibitor, such as the anti-PD-1 antibody molecule. For example, inhibition of an activity, e.g., a PD-1 or PD-L1 activity, of at least 5%, 10%, 20%, 30%, 40% or more is included by this term. Thus, inhibition need not be 100%.

The term “cancer” refers to a disease characterized by the rapid and uncontrolled growth of aberrant cell proliferation. Cancer cells can spread locally or through the bloodstream and lymphatic system to other pails of the body. Examples of various cancers are, but are not limited to, leukemia, prostate cancer, renal cancer, liver cancer, sarcoma, brain cancer, lymphoma, ovarian cancer, lung cancer, cervical cancer, skin cancer, breast cancer, head and neck squamous cell carcinoma (HNSCC), pancreatic cancer, gastrointestinal cancer, colorectal cancer, triple-negative breast cancer (TNBC), squamous cell cancer of the lung, squamous cell cancer of the esophagus, squamous cell cancer of the cervix, gliomas, glioblastomas, or melanoma. According to the disclosure the particularly amenable disease conditions to be treated with the aforementioned combination is glioblastoma multiforme (GBM).

The terms “tumor” and “cancer” are used interchangeably herein, e.g., both terms encompass solid and liquid, e.g., diffuse or circulating tumors. In one embodiment, the term “cancer” or “tumor” includes malignant cancers and tumors, as well as advanced cancers and tumors.

The term “treatment” comprises, for example, the therapeutic administration of the combination of a CSF-1R inhibitor, or a pharmaceutically acceptable salt thereof, and an anti-PD-1 antibody molecule, or a pharmaceutically acceptable salt thereof, as described herein to a warm-blooded animal, in particular a human being, in need of such treatment with the aim to cure the disease or to have an effect on disease regression or on the delay of progression of a disease. The terms “treat”, “treating” or “treatment” of any disease or disorder refers to ameliorating the disease or disorder (e.g. slowing or arresting or reducing the development of the disease or at least one of the clinical symptoms thereof), to preventing or delaying the onset or development or progression of the disease or disorder.

A CSF-1R inhibitor, (i) 4-((2-(((1R,2R)-2-hydroxycyclohexyl)amino)benzo[d]thiazol-6-yl)oxy)-N-methylpicolinamide, or a pharmaceutically acceptable salt thereof can be administered 4 days-on and 10 days-off, twice per cycle. The CSF-1R inhibitor as disclosed herein can be administered once daily or twice daily with a 12-hour gap between two consecutive doses. The combination partner (ii) an anti-PD-1 antibody molecule can continue to be administered for more cycles as long as it is clinically meaningful.

The combination partners, as disclosed herein, are administered on the same day or on different days of a cycle. The term “cycle” refers to a specific period of time expressed in days or months that is repeated on a regular schedule. The cycle as disclosed herein is more preferably expressed in days. For example, the cycle can be, but is not limited to, 28 days, 30 days, 60 days, 90 days. Most preferably, the “cycle” as referred to in the present disclosure is 28 days long. Such cycle can be repeated several time (e.g. 2 times, 3 times, 4 times, 5 times, etc.), each cycle being the same length and can be repeated as long as it is clinically meaningful, i.e. the tumor growth is at least reduced, or controlled, or the tumor shrinks, and the adverse events are tolerable.

The CSF-1R inhibitor (i) 4-((2-(((R,2R)-2-hydroxycyclohexyl)amino)benzo[d]thiazol-6-yl)oxy)-N-methylpicolinamide, or a pharmaceutically acceptable salt thereof, can be administered orally or intravenously, most preferably orally, at a daily dose of 100 mg/day, 150 mg/day, 200 mg/day, 300 mg/day, 400 mg/day. 500 mg/day, 600 mg/day, 700 mg/day, 900 mg/day, or 1200 mg/day. Preferably, the daily dose is 700 mg/day, or 1200 mg/day.

According to the present disclosure (i) 4-((2-(((1R,2R)-2-hydroxycyclohexyl)amino)benzo[d]thiazol-6-yl)oxy)-N-methylpicolinamide, or a pharmaceutically acceptable salt thereof, can be administered orally, for example, in a pharmaceutical composition together with an inert diluent or carrier.

In accordance with the present disclosure the anti-PD-1 antibody molecule (ii), or a pharmaceutically acceptable salt thereof, selected from nivolumab (Opdivo), pembrolizumab (Keytruda), pidilizumab, spartalizumab, or a pharmaceutical salt thereof, can be used in the treatment of cancer, and is administered every two weeks or every four weeks in a cycle. Most preferably the anti-PD-1 antibody molecule spartalizumab (ii), or a pharmaceutically acceptable salt thereof, as described herein, used in the treatment of cancer. Most preferably spartalizumab (ii) is administered every four weeks. Spartalizumab is administered by injection (e.g. subcutaneously or intravenously) at a dose of 300-400 mg/day. Preferably, the anti-PD-1 antibody molecule spartalizumab, or a pharmaceutically acceptable salt thereof, is administered intravenously in a single dose of 300 to 400 mg/day. Most preferably, the anti-PD-1 antibody molecule spartalizumab (ii), or a pharmaceutically acceptable salt thereof, is administered in a single dose of 400 mg/day. Most preferably, the anti-PD-1 antibody molecule spartalizumab, or a pharmaceutically acceptable salt thereof, is administered at a dose of 400 mg/day every four weeks. The dose can be administered in a single dose or in several divided doses.

Specifically, the dosing schedule can vary from 100 mg/day. 150 mg/day. 200 mg/day. 300 mg/day. 400 mg/day. 500 mg/day, 600 mg/day, 700 mg/day, 900 mg/day, or 1200 mg/day of CSF-1R inhibitor of Formula (I) 4-((2-(((1R,2R)-2-hydroxycyclohexyl)amino)benzo[d]thiazol-6-yl)oxy)-N-methylpicolinamide, or a pharmaceutically acceptable salt thereof (4 days-on and 10 days-off, twice per cycle) and from 300 mg/day to 400 mg/day of anti-PD-1 antibody molecule (ii), or a pharmaceutically acceptable salt thereof, every two or four weeks. For example, according to the present disclosure, 150 mg/day of (i) 4-((2-(((1R,2R)-2-hydroxycyclohexyl)amino)benzo[d]thiazol-6-yl)oxy)-N-methylpicolinamide, or a pharmaceutically acceptable salt thereof, is 4 days-on and 10 days-off, twice per cycle, and anti-PD-1 antibody molecule (ii), or a pharmaceutically acceptable salt thereof, is administered once every 4 weeks per cycle at a dose of 400 mg/day. Another example, according to the present disclosure, consists of administering 300 mg/day of (i) 4-((2-(((1R,2R)-2-hydroxycyclohexyl)amino)benzo[d]thiazol-6-yl)oxy)-N-methylpicolinamide, or a pharmaceutically acceptable salt thereof, 4 days-on and 10 days-off, twice per cycle, and administering anti-PD-1 antibody molecule (ii), or a pharmaceutically acceptable salt thereof, once every 4 weeks per cycle at a dose of 400 mg/day. Yet another example, according to the present disclosure, provides the administration of 600 mg/day of (i) 4-((2-(((1R,2R)-2-hydroxycyclohexyl)amino)benzo[d]thiazol-6-yl)oxy)-N-methylpicolinamide, or a pharmaceutically acceptable salt thereof 4 days-on and 10 days-off, twice per cycle, and anti-PD-1 antibody molecule (ii), or a pharmaceutically acceptable salt thereof, is administered once every 4 weeks per cycle at a dose of 400 mg/day.

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 300 to 400 mg/day. For intravenous injection or infusion, therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients as required, followed by filtered sterilization. Generally, dispersions are prepared by incorporating the active compound into a sterile vehicle that contains a basic dispersion medium and the required other ingredients. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

It would be understood that the route and/or mode of administration will vary depending upon the desired results. For example, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (e.g., Sustained and Controlled Release Drug Delivery Systems, w. R. Robinson, ed., Marcel Dekker, Inc., New York, 1978).

Equally, (i) 4-((2-(((1R,2R)-2-hydroxycyclohexyl)amino)benzo[d]thiazol-6-yl)oxy)-N-methylpicolinamide, or a pharmaceutically acceptable salt thereof, in combination with anti-PD-1 antibody molecule (ii), or a pharmaceutically acceptable salt, can be used for the manufacture of a medicament for the treatment of cancer.

By the same token, the present disclosure also provides a method for the treatment of cancer, comprising administering an effective amount of the combination partners (e.g. (i) 4-((2-(((1R,2R)-2-hydroxycyclohexyl)amino)benzo[d]thiazol-6-yl)oxy)-N-methylpicolinamide, or a pharmaceutically acceptable salt thereof and anti-PD-1 antibody molecule (ii), or a pharmaceutically acceptable salt thereof) to a patient in need thereof.

The term “patient” or “subject” refers to a warm-blooded animal. In a most preferred embodiment, the subject or patient is human. It may be a human who has been diagnosed and is in the need of treatment for a disease or disorder, as disclosed herein.

When used for the manufacture of a medicament for the treatment of cancer or in a method of treating a cancer in a patient in need thereof, (i) and (ii) can be used in doses and dosing schedules as explained above.

Most preferably the combination comprises the CSF-1R inhibitor (i) 4-((2-(((1R,2R)-2-hydroxycyclohexyl)amino)benzo[d]thiazol-6-yl)oxy)-N-methylpicolinamide, or a pharmaceutically acceptable salt thereof, and anti-PD-1 antibody molecule spartalizumab (ii), or a pharmaceutically acceptable salt thereof. Both combination partners (i) and (ii) can be administered according to the doing schedule as described herein. For example (i) 4-((2-(((1R,2R)-2-hydroxycyclohexyl)amino)benzo[d]thiazol-6-yl)oxy)-N-methylpicolinamide, or a pharmaceutically acceptable salt thereof, can be administered 4 days-on and 10 days-off, twice per cycle. The spartalizumab (ii), or a pharmaceutically acceptable salt thereof, is administered at least once per cycle. For example, (i) 4-((2-(((1R,2R)-2-hydroxycyclohexyl)amino)benzo[d]thiazol-6-yl)oxy)-N-methylpicolinamide, or a pharmaceutically acceptable salt thereof is administered in this specific combination at a dose of 100 mg/day, 150 mg/day, 200 mg/day, 300 mg/day, 400 mg/day, 500 mg/day, 600 mg/day, 700 mg/day, 900 mg/day, or 1200 mg/day. Preferably, the dose is 700 mg/day or 1200 mg/day. Spartalizumab (ii), or a pharmaceutically acceptable salt thereof, is administered in a single dose of 300-400 mg/day, most preferably a dose of 400 mg/day.

By the same token, the present disclosure also provides a method for the treatment of cancer, comprising administering an effective amount of the combination partners (e.g. (i) 4-((2-(((1R,2R)-2-hydroxycyclohexyl)amino)benzo[d]thiazol-6-yl)oxy)-N-methylpicolinamide, or a pharmaceutically acceptable salt thereof and anti-PD-1 antibody molecule (ii), or a pharmaceutically acceptable salt thereof) to a patient in need thereof.

The term “effective amount” or “therapeutically effective amount” of the combination partners of the present disclosure, refers to an amount effective, at dosages and for periods of time necessary, to achieve the desired therapeutic result. A therapeutically effective amount of the combination partners may vary according to factors such as the disease state, age, sex, and weight of the individual. A therapeutically effective amount is also one in which any toxic or detrimental effects of the combination, as described herein, is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects.

TABLE 1
Amino acid and nucleotide sequences for humanized antibody molecules. The antibody molecules include
BAP049-Clone-B and BAP049-Clone-E. The amino acid and nucleotide sequences of the heavy and light
chain CDRs, the heavy and light chain variable regions, and the heavy and light chains are shown.
BAP049-Clone-B HC

| | | |
|---|---|---|
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFT |
| | | TYWMHVWRQATGQGLEWMGNIYPGTGGS |
| | | NFDEKFKNRVTITADKSTSTAYMELSSLRSE |
| | | DTAVYYCTRWTTGTGAYWGQGTTVTVSS |
| SEQ ID NO: 95 | DNA VH | GAGGTGCAGCTGGTGCAGTCAGGCGCCG |
| | | AAGTGAAGAAGCCCGGCGAGTCACTGAG |
| | | AATTAGCTGTAAAGGTTCAGGCTACACCT |
| | | TCACTACCTACTGGATGCACTGGGTCCGC |
| | | CAGGCTACCGGTCAAGGCCTCGAGTGGA |
| | | TGGGTAATATCTACCCCGGCACCGGCGG |
| | | CTCTAACTTCGACGAGAAGTTTAAGAATA |
| | | GAGTGACTATCACCGCCGATAAGTCTACT |
| | | AGCACCGCCTATATGGAACTGTCTAGCCT |
| | | GAGATCAGAGGACACCGCCGTCTACTACT |
| | | GCACTAGGTGGACTACCGGCACAGGCGC |
| | | CTACTGGGGTCAAGGCACTACCGTGACC |
| | | GTGTCTAGC |
| SEQ ID NO: 91 | HC | EVQLVQSGEVKKPGESLRISCKGSGYTFTT |
| | | TYWMHVWRQATGQGLEWMGNIYPGTGGS |
| | | NFDEKFKNRVTITADKSTSTAYMELSSLRSE |
| | | DTAVYYCTRWTTGTGAYWGQGTTVTVSSA |
| | | STKGPSVFPLAPCSRSTSESTAALGCLVKD |
| | | YFPEPVTVSWNSGALTSGVHTFPAVLQSSG |
| | | LYSLSSVVTVPSSSLGTKTYTCNVDHKPSN |
| | | TKVDKRVESKYGPPCPPCPAPEFLGGPSVF |
| | | LFPPKPKDTLMISRTPEVTCVVVDVSQEDP |
| | | EVQFNWYVDGVEVHNAKTKPREEQFNSTY |
| | | RVVSVLTVLHQDWLNGKEYKCKVSNKGLP |
| | | SSIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| SEQ ID NO: 96 | DNA HC | GAGGTGCAGCTGGTGCAGTCAGGCGCCG |
| | | AAGTGAAGAAGCCCGGCGAGTCACTGAG |
| | | AATTAGCTGTAAAGGTTCAGGCTACACCT |
| | | TCACTACCTACTGGATGCACTGGGTCCGC |
| | | CAGGCTACCGGTCAAGGCCTCGAGTGGA |
| | | TGGGTAATATCTACCCCGGCACCGGCGG |
| | | CTCTAACTTCGACGAGAAGTTTAAGAATA |
| | | GAGTGACTATCACCGCCGATAAGTCACT |
| | | AGCACCGCCTATATGGAACTGTCTAGCCT |
| | | GAGATCAGAGGACACCGCCGTCTACTACT |
| | | GCACTAGGTGGACTACCGGCACAGGCGC |
| | | CTACTGGGGTCAAGGCACTACCGTGACC |
| | | GTGTCTAGCGCTAGCACTAAGGGCCCGT |
| | | CCGTGTTCCCCCTGGCACCTTGTAGCCG |
| | | GAGCACTAGCGAATCCACCGCTGCCCTC |
| | | GGCTGCCTGGTCAAGGATTACTTCCCGG |
| | | AGCCCGTGACCGTGTCCTGGAACAGCGG |
| | | AGCCCTGACCTCCGGAGTGCACACCTTC |
| | | CCCGCTGTGCTGCAGAGCTCCGGGCTGT |
| | | ACTCGCTGTCGTCGGTGGTCACGGTGCC |
| | | TTCATCTAGCCTGGGTACCAAGACCTACA |
| | | CTTGCAACGTGGACCACAAGCCTTCCAAC |
| | | ACTAAGGTGGACAAGCGCGTCGAATCGA |
| | | AGTACGGCCCACCGTGCCCGCCTTGTCC |
| | | CGCGCCGGAGTTCCTCGGCGGTCCCTCG |
| | | GTCTTTCTGTTCCCACCGAAGCCCAAGGA |
| | | CACTTTGATGATTTCCCGCACCCCTGAAG |
| | | TGACATGCGTGGTCGTGGACGTGTCACA |
| | | GGAAGATCCGGAGGTGCAGTTCAATTGG |
| | | TACGTGGATGGCGTCGAGGTGCACAACG |
| | | CCAAAACCAAGCCGAGGGAGGAGCAGTT |
| | | CAACTCCACTTACCGCGTCGTGTCCGTGC |
| | | TGACGGTGCTGCATCAGGACTGGCTGAA |
| | | CGGGAAGGAGTACAAGTGCAAAGTGTCC |
| | | AACAAGGGACTTCCTAGCTCAATCGAAAA |
| | | GACCATCTCGAAAGCCAAGGGACAGCCC |
| | | CGGGAACCCCAAGTGTATACCCTGCCAC |
| | | CGAGCCAGGAAGAAATGACTAAGAACCAA |
| | | GTCTCATTGACTTGCCTTGTGAAGGGCTT |
| | | CTACCCATCGGATATCGCCGTGGAATGG |
| | | GAGTCCAACGGCCAGCCGGAAAACAACT |
| | | ACAAGACCACCCCTCCGGTGCTGGACTC |
| | | AGACGGATCCTTCTTCCTCTACTCGCGGC |
| | | TGACCGTGGATAAGAGCAGATGGCAGGA |
| | | GGGAAATGTGTTCAGCTGTTCTGTGATGC |
| | | ATGAAGCCCTGCACAACCACTACACTCAG |
| | | AAGTCLCCTGTCCCTCTCCCTGGGA |

BAP049-Clone-B LC

| | | |
|---|---|---|
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SeQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 54 | VL | EIVLTQSPTLSLSPGERTLSCKSSQSLLD |
| | | SGNQKNFLTWYQQKPGKAPKLLIYWASTR |
| | | ESGVPSRFSGSGSGTDFTFTISSLQPEDIAT |
| | | YYCQNDYSYPYTFGQGTKVEIK |
| SEQ ID NO: 97 | DNA VL | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTAAA |
| | | GCCCCTAAGCTGCTGATCTACTGGGCCTC |
| | | TACTAGAGAATCAGGCGTGCCCTCTAGGT |
| | | TTAGCGGTAGCGGTAGTGGCACCGACTT |
| | | CACCTTCACTATCTCTAGCCTGCAGCCCG |
| | | AGGATATCGCTACCTACTACTGTCAGAAC |
| | | GACTATAGCTACCCCTACACCTTCGGTCA |
| | | AGGCACTAAGGTCGAGATTAAG |
| SEQ ID NO: 56 | LC | EIVLTQSPATLSLSPGERATLSCKSSQSLLD |
| | | SGNQKNFLTWYQQKPGKAPKLLIYWASTR |
| | | ESGVHSHFSGSGSGTCFTFTISSLQPEDIAT |
| | | YYCQNDYSYPYTFGQGTKVEIKRTVAAPSV |
| | | FIFPPSDEQLKSGTASVVCLLNNFYPREAKV |
| | | QWKVDNALQSGNSQESVTEQDSKDSTYSL |
| | | SSTLTLSKADYEKHKVYACEVTHQGLSSPV |
| | | TKSFNRGEC |
| SEQ ID NO: 98 | DNA LC | GAGATCGTCCTGALTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTAAA |
| | | GCCCCTAAGCTGCTGATCTACTGGGCCTC |
| | | TACTAGAGAATCAGGCGTGCCCTCTAGGT |
| | | TTAGCGGTAGCGGTAGTGGCACCGACTT |
| | | CACCTTCACTATCTCTAGCCTGCAGCCCG |
| | | AGGATATCGCTACCTACTACTGTCAGAAC |
| | | GACTATAGCTACCCCTACACCTTCGGTCA |
| | | AGGCACTAAGGTCGAGATTAAGCGTACG |
| | | GTGGCCGCTCCCAGCGTGTTCATCTTCCC |
| | | CCCCAGCGACGAGCAGCTGAAGAGCGGC |
| | | ACCGCCAGCGTGGTGTGCCTGCTGAACA |
| | | ACTTCTACCCCCGGGAGGCCAAGGTGCA |
| | | GTGGAAGGTGGACAACGCCCTGCAGAGC |
| | | GGCAACAGCCAGGAGAGCGTCACCGAGC |
| | | AGGACAGCAAGGACTCCACCTACAGCCT |
| | | GAGCAGCACCCTGACCCTGAGCAAGGCC |
| | | GACTACGAGAAGCATAAGGTGTACGCCT |
| | | GCGAGGTGACCCACCAGGGCCTGTCCAG |
| | | CCCCGTGACCAAGAGCTTCAACAGGGGC |
| | | GAGTGC |
| SEQ ID NO: 92 | DNA HC | GAAGTGCAGCTGGTGCAGTCTGGCGCCG |
| | | AAGTGAAGAAGCCTGGCGAGTCCCTGCG |
| | | GATCTCCTGCAAGGGCTCTGGCTACACCT |
| | | TCACCACCTACTGGATGCACTGGGTGCG |
| | | ACAGGCTACCGGCCAGGGCCTGGAATGG |
| | | ATGGGCAACATCTATCCTGGCACCGGCG |
| | | GCTCCAACTTCGACGAGAAGTTCAAGAAC |
| | | AGAGTGACCATCACCGCCGACAAGTCCA |
| | | CCTCCACCGCCTACATGGAACTGTCCTCC |
| | | CTGAGATCCGAGGACACCGCCGTGTACT |
| | | ACTGCACCCGGTGGACAACCGGCACAGG |
| | | CGCTTATTGGGGCCAGGGCACCACAGTG |
| | | ACCGTGTCCTCTGCTTCTACCAAGGGGCC |
| | | CAGCGTGTTCCCCCTGGCCCCCTGCTCC |
| | | AGAAGCACCAGCGAGAGCACAGCCGCCC |
| | | TGGGCTGCCTGGTGAAGGACTACTTCCC |
| | | CGAGCCCGTGACCGTGTCCTGGAACAGC |
| | | GGAGCCCTGACCAGCGGCGTGCACACCT |
| | | TCCCCGCCGTGCTGCAGAGCAGCGGCCT |
| | | GTACAGCCTGAGCAGCGTGGTGACCGTG |
| | | CCCAGCAGCAGCCTGGGCACCAAGACCT |
| | | ACACCTGTAACGTGGACCACAAGCCCAG |
| | | CAACACCAAGGTGGACAAGAGGGTGGAG |
| | | AGCAAGTACGGCCCACCCTGCCCCCCCT |
| | | GCCCAGCCCCCGAGTTCCTGGGCGGACC |
| | | CAGCGTGTTCCTGTTCCCCCCCAAGCCCA |
| | | AGGACACCCTGATGATCAGCAGAACCCC |
| | | CGAGGTGACCTGTGTGGTGGTGGACGTG |
| | | TCCCAGGAGGACCCCGAGGTCCAGTTCA |
| | | ACTGGTACGTGGACGGCGTGGAGGTGCA |
| | | CAACGCCAAGACCAAGCCCAGAGAGGAG |
| | | CAGTTTAACAGCACCTACCGGGTGGTGTC |
| | | CGTGCTGACCGTGCTGCACCAGGACTGG |
| | | CTGAACGGCAAAGAGTACAAGTGTAAGGT |
| | | CTCCAACAAGGGCCTGCCAAGCAGCATC |
| | | GAAAAGACCATCAGCAAGGCCAAGGGCC |
| | | AGCCTAGAGAGCCCCAGGTCTACACCCT |
| | | GCCACCCAGCCAAGAGGAGATGACCAAG |
| | | AACCAGGTGTCCCTGACCTGTCTGGTGAA |
| | | GGGCTTCTACCCAAGCGACATCGCCGTG |
| | | GAGTGGGAGAGCAACGGCCAGCCCGAGA |
| | | ACAACTACAAGACCACCCCCCCAGTGCTG |
| | | GACAGCGACGGCAGCTTCTTCCTGTACA |
| | | GCAGGCTGACCGTGGACAAGTCCAGATG |
| | | GCAGGAGGGCAACGTCTTTAGCTGCTCC |
| | | GTGATGCACGAGGCCCTGCACAACCACT |
| | | ACACCCAGAAGAGCCTGAGCCTGTCCCT |
| | | GGGC |

BAP049-Clone-E LC

| | | |
|---|---|---|
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLD |
| | | SGNQKNFLTWYQQKPGQAPRLLIYWASTR |
| | | ESGVPSRFSGSGSGTDFTFTISSLEAEDAA |
| | | TYYCQNDYSYPYTFGQGTKVEIK |
| SEQ ID NO: 106 | DNA VL | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTCA |
| | | AGCCCCTAGACTGCTGATCTACTGGGCCT |
| | | CTACTAGAGAATCAGGCGTGCCCTCTAGG |
| | | TTTAGCGGTAGCGGTAGTGGCACCGACTT |
| | | CACCTTCACTATCTCTAGCCTGGAAGCCG |
| | | AGGACGCCGCTACCTACTACTGTCAGAAC |
| | | GACTATAGCTACCCCTACACCTTCGGTCA |
| | | AGGCACTAAGGTCGAGATTAAG |
| SEQ ID NO: 72 | LC | EIVLTQSPATLSLSPGERATLSCKSSQSLLD |
| | | SGNQKNFLTWYQQKPGQAPRLLIYWASTR |
| | | ESGVPSRFSGSGSGTDFTFTISSLEAEDAA |
| | | TYYCQNDYSYPYTFGQGTKVEIKRTVAAPS |
| | | VFIFPPSDEQLKSGTASWCLLNNFYPREAK |
| | | VQWKVDNALQSGNSQESVTEQDSKDSTYS |
| | | LSSTLTLSKADYEKHKVYACEVTHQGLSSP |
| | | VTKSFNRGEC |
| SEQ ID NO: 107 | DNA LC | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTCA |
| | | AGCCCCTAGACTGCTGATCTACTGGGCCT |
| | | CTACTAGAGAATCAGGCGTGCCCTCTAGG |
| | | TTTAGCGGTAGCGGTAGTGGCACCGACTT |
| | | CACCTTCACTATCTCTAGCCTGGAAGCCG |
| | | AGGACGCCGCTACCTACTACTGTCAGAAC |
| | | GACTATAGCTACCCCTACACCTTCGGTCA |
| | | AGGCACTAAGGTCGAGATTAAGCGTACG |
| | | GTGGCCGCTCCCAGCGTGTTCATCTTCCC |
| | | CCCCAGCGACGAGCAGCTGAAGAGCGGC |
| | | ACCGCCAGCGTGGTGTGCCTGCTGAACA |
| | | ACTTCTACCCCCGGGAGGCCAAGGTGCA |
| | | GTGGAAGGTGGACAACGCCCTGCAGAGC |
| | | GGCAACAGCCAGGAGAGCGTCACCGAGC |
| | | AGGACAGCAAGGACTCCACCTACAGCCT |
| | | GAGCAGCACCCTGACCCTGAGCAAGGCC |
| | | GACTACGAGAAGCATAAGGTGTACGCCT |
| | | GCGAGGTGACCCACCAGGGCCTGTCCAG |
| | | CCCCGTGACCAAGAGCTTCAACAGGGGC |
| | | GAGTGC |

BAP049-Clone-B HC

| | | |
|---|---|---|
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAA |
| | | CTTCGACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |

BAP049-Clone-B LC

| | | |
|---|---|---|
| SEQ ID NO: 138 (Kabat) | LCDR1 | AAATCTAGTCGTCACTGCTGGATAGCGGT |
| | | TAATCAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCA |
| | | GAAGAACTTC |
| SEQ ID NO: 142 (Chothia) | LCDR2 | TGGGCCTCT |
| SEQ ID NO: 143 (Chothia) | LCDR3 | GACTATAGCTACCCCTAC |

BAP049-Clone-E HC

| | | |
|---|---|---|
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAA |
| | | CTTCGACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |

BAP049-Clone-E LC

| | | |
|---|---|---|
| SEQ ID NO: 138 (Kabat) | LCDR1 | AAATCTAGTCAGTCACTGCTGGATAGCGG |
| | | TAATCAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCA |
| | | GAAGAACTTC |
| SEQ ID NO: 142 (Chothia) | LCDR2 | TGGGCCTCT |
| SEQ ID NO: 143 (Chothia) | LCDR3 | GACTATAGCTACCCCTAC |

TABLE 2
Amino acid and nucleotide sequences of the heavy and light chain framework regions for humanized
mAbs BAP049-Clone-B and BAP049-Clone-E

| | Amino Acid Sequence | Nucleotide Sequence |
|---|---|---|
| VHFW1 | EVQLVQSGAEVKKPGESLRISCKGS | GAAGTGCAGCTGGTGCAGTCTGGAGCAGA |
| (type a) | (SEQ ID NO: 147) | GGTGAAAAAGCCCGGGGAGTCTCTGAGGAT |
| | | CTCCTGTAAGGGTTCT (SEQ ID NO: 148) |
| | | GAAGTGCAGCTGGTGCAGTCTGGCGCCGA |
| | | AGTGAAGAAGCCTGGCGAGTCCCTGCGGAT |
| | | CTCCTGCAAGGGCTCT (SEQ ID NO: 149) |
| | | GAGGTGCAGCTGGTGCAGTCAGGCGCCGA |
| | | AGTGAAGAAGCCCGGCGAGTCACTGAGAAT |
| | | TAGCTGTAAAGGTTCA (SEQ ID NO: 150) |
| VHFW1 | QVQLVQSGAEVKKPGASVKVSCKA | CAGGTTCAGCTGGTGCAGTCTGGAGCTGAG |
| (type b) | S (SEQ ID NO: 151) | GTGAAGAAGCCTGGGGCCTCAGTGAAGGTC |
| | | TCCTGCAAGGCTTCT (SEQ ID NO: 152) |
| VHFW2 | WVRQATGQGLEWMG | TGGGTGCGACAGGCCACTGGACAAGGGCT |
| (type a) | (SEQ ID NO: 153) | TGAGTGGATGGGT (SEQ ID NO: 154) |
| | | TGGGTGCGACAGGCTACCGGCCAGGGCCT |
| | | GGAATGGATGGGC (SEQ ID NO: 155) |
| | | TGGGTCCGCCAGGCTACCGGTCAAGGCCT |
| | | CGAGTGGATGGGT (SEQ ID NO: 156) |
| VHFW2 | WIRQSPSRGLEWLG | TGGATCAGGCAGTCCCCATCGAGAGGCCTT |
| (type b) | (SEQ ID NO: 157) | GAGTGGCTGGGT (SEQ ID NO: 158) |
| | | TGGATCCGGCAGTCCCCCTCTAGGGGCCTG |
| | | GAATGGCTGGGC (SEQ ID NO: 159) |
| VHFW2 | WVRQAPGQGLEWMG | TGGGTGCGACAGGCCCCTGGACAAGGGCT |
| (type c) | (SEQ ID NO: 160) | TGAGTGGATGGGT (SEQ ID NO: 161) |
| VHFW3 | RVTITADKSTSTAYMELSSLRSEDTA | AGAGTCACGATTACCGCGGACAAATCCACG |
| (type a) | VYYCTR (SEQ ID NO: 162) | AGCACAGCCTACATGGAGCTGAGCAGCCTG |
| | | AGATCTGAGGACACGGCCGTGTATTACTGT |
| | | ACAAGA (SEQ ID NO: 163) |
| | | AGAGTGACCATCACCGCCGACAAGTCCACC |
| | | TCCACCGCCTACATGGAACTGTCCTCCCTG |
| | | AGATCCGAGGACACCGCCGTGTACTACTGC |
| | | ACCCGG (SEQ ID NO: 164) |
| | | AGAGTGACTATCACCGCCGATAAGTCTACTA |
| | | GCACCGCCTATATGGAACTGTCTAGCCTGA |
| | | GATCAGAGGACACCGCCGTCTACTACTGCA |
| | | CTAGG (SEQ ID NO: 165) |
| VHFW3 | RFTISRDNSKNTLYLQMNSLRAEDT | AGATTCACCATCTCCAGAGACAATTCCAAGA |
| (type b) | AVYYCTR (SEQ ID NO: 166) | ACACGCTGTATCTTCAAATGAACAGCCTGAG |
| | | AGCCGAGGACACGGCCGTGTATTACTGTAC |
| | | AAGA (SEQ ID NO: 167) |
| | | AGGTTCACCATCTCCCGGGACAACTCCAAG |
| | | AACACCCTGTACCTGCAGATGAACTCCCTG |
| | | CGGGCCGAGGACACCGCCGTGTACTACTGT |
| | | ACCAGA (SEQ ID NO: 168) |
| VHFW4 | WGQGTTVTVSS | TGGGGCCAGGGCACCACCGTGACCGTGTC |
| | (SEQ ID NO: 169) | CTCC (SEQ ID NO. 170) |
| | | TGGGGCCAGGGCACCACAGTGACCGTGTC |
| | | CTCT (SEQ ID NO: 171) |
| | | TGGGGTCAAGGCACTACCGTGACCGTGTCT |
| | | AGC (SEQ ID NO: 172) |
| | | TGGGGCCAGGGCACAACAGTGACCGTGTC |
| | | CTCC (SEQ ID NO: 173) |
| VLFW1 | EIVLTQSPDFQSVTPKEKVTITC | GAAATTGTGCTGACTCAGTCTCCAGACTTTC |
| (type a) | (SEQ ID NO: 174) | AGTCTGTGACTCCAAAGGAGAAAGTCACCA |
| | | TCACCTGC (SEQ ID NO: 175) |
| | | GAGATCGTGCTGACCCAGTCCCCCGACTTC |
| | | CAGTCCGTGACCCCCAAAGAAAAAGTGACC |
| | | ATCACATGC (SEQ ID NO: 176) |
| VLFW1 | EIVLTQSPATLSLSPGERATLSC | GAAATTGTGTTGACACAGTCTCCAGCCACC |
| (type b) | (SEQ ID NO: 177) | CTGTCTTTGTCTCCAGGGGAAAGAGCCACC |
| | | CTCTCCTGC (SEQ ID NO: 178) |
| | | GAGATCGTGCTGACCCAGTCCCCTGCCACC |
| | | CTGTCACTGTCTCCAGGCGAGAGAGCTACC |
| | | CTGTCCTGC (SEQ ID NO: 179) |
| | | GAGATCGTCCTGACTCAGTCACCCGCTACC |
| | | CTGAGCCTGAGCCCTGGCGAGLGGGCTAC |
| | | ACTGAGCTGT (SEQ ID NO: 180) |
| VLFW1 | DIVMTQTPLSLPVTPGEPASISC | GATATTGTGATGACCCAGACTCCACTCTCCC |
| (type c) | (SEQ ID NO: 181) | TGCCCGTCACCCCTGGAGAGCCGGCCTCC |
| | | ATCTCCTGC (SEQ ID NO: 182) |
| VLFW1 | DVVMTQSPLSLPVTLGQPASISC | GATGTTGTGATGACTCAGTCTCCACTCTCCC |
| (type d) | (SEQ ID NO: 183) | TGCCCGTCACCCTTGGACAGCCGGCCTCCA |
| | | TCTCCTGC (SEQ ID NO: 184) |
| VLFW* | DIQMTQSPSSLSASVGDRVTITC | GACATCCAGATGACCCAGTCTCCATCCTCC |
| (type e) | (SEQ ID NO: 185) | CTGTCTGCATCTGTAGGAGACAGAGTCACC |
| | | ATCACTTGC (SEQ ID NO: 186) |
| VLFW2 | WYQQKPGQAPRLLIY | TGGTACCAGCAGAAACCTGGCCAGGCTCCC |
| (type a) | (SEQ ID NO: 187) | AGGCTCCTCATCTAT (SEQ ID NO: 188) |
| | | TGGTATCAGCAGAAGCCCGGCCAGGCCCC |
| | | CAGACTGCTGATCTAC (SEQ ID NO: 189) |
| | | TGGTATCAGCAGAAGCCCGGTCAAGCCCCT |
| | | AGACTGCTGATCTAC (SEQ ID NO: 190) |
| VLFW2 | WQQKPGKAPKLLIY | TGGTATCAGCAGAAACCAGGGAAAGCTCCT |
| (type b) | (SEQ ID NO: 191) | AAGCTCCTGATCTAT (SEQ ID NO: 192) |
| | | TGGTATCAGCAGAAGCCCGGTAAAGCCCCT |
| | | AAGCTGCTGATCTAC (SEQ ID NO: 193) |
| VLFW2 | WYLQKPGQSPQLLIY | TGGTACCTGCAGAAGCCAGGGCAGTCTCCA |
| (type c) | (SEQ ID NO: 194) | CAGCTCCTGATCTAT (SEQ ID NO: 195) |
| VLFW3 | GVPSRFSGSGSGTDFTFTISSLEAE | GGGGTCCCCTCGAGGTTCAGTGGCAGTGG |
| (type a) | DAATYYC (SEQ ID NO: 196) | ATCTGGGACAGATTTCACCTTTACCATCAGT |
| | | AGCCGGAAGCTGAAGATGCTGCAACATAT |
| | | TACTGT (SEQ ID NO: 197) |
| | | GGCGTGCCCTCTAGATTCTCCGGCTCCGGC |
| | | TCTGGCACCGACTTTACCTTCACCATCTCCA |
| | | GCCTGGAAGCCGAGGACGCCGCCACCTAC |
| | | TACTGC (SEQ ID NO: 198) |
| | | GGCGTGCCCTCTAGGTTTAGCGGTAGCGGT |
| | | AGTGGCACCGACTTCACCTTCACTATCTCTA |
| | | GCCTGGAAGCCGAGGACGCCGCTACCTACT |
| | | ACTGT (SEQ ID NO: 199) |
| VLFW3 | GIPPRFSGSGYGTDFTLTINNIESED | GGGATCCCACCTCGATTCAGTGGCAGCGG |
| (type b) | AAYYFC (SEQ ID NO: 200) | GTATGGAACAGATTTTACCCTCACAATTAAT |
| | | AACATAGAATCTGAGGATGCTGCATATTACT |
| | | TCTGT (SEQ ID NO: 201) |
| VLFW3 | GVPSRFSGSGSGTEFTLTISSLQPD | GGGGTCCCATCAAGGTTCAGCGGCAGTGG |
| (type c) | DFATYYC (SEQ ID NO: 202) | ATCTGGGACAGAATTCACTCTCACCATCAGC |
| | | AGCCTGCAGCCTGATGATTTTGCAACTTATT |
| | | ACTGT (SEQ ID NO: 203) |
| | | GGCGTGCCCTCTAGATTCTCCGGCTCCGGC |
| | | TCTGGCACCGAGTTTACCCTGACCATCTCC |
| | | AGCCTGCAGCCCGACGACTTCGCCACCTAC |
| | | TACTGC (SEQ ID NO: 204) |
| VLFW3 | GVPSRFSGSGSGTDFTFTISSLQPE | GGGGTCCCATCAAGGTTCAGTGGAAGTGGA |
| (type d) | DIATYYC (SEQ ID NO: 205) | TCTGGGACAGATTTTACTTTCACCATCAGCA |
| | | GCCTGCAGCCTGAAGATATTGCAACATATTA |
| | | CTGT (SEQ ID NO: 206) |
| | | GGCGTGCCCTCTAGGTTTAGCGGTAGCGGT |
| | | AGTGGCACCGACTTCACCTTCACTATCTCTA |
| | | GCCTGCAGCCCGAGGATATCGCTACCTACT |
| | | ACTGT (SEQ ID NO: 207) |
| VLFW4 | FGQGTKVEIK (SEQ ID NO: 208) | TTCGGCCAAGGGACCAAGGTGGAAATCAAA |
| | | (SEQ ID NO: 209) |
| | | TTCGGCCAGGGCACCAAGGTGGAAATCAAG |
| | | (SEQ ID NO: 210) |
| | | TTCGGTCAAGGCACTAAGGTCGAGATTAAG |
| | | (SEQ ID NO: 211) |

TABLE 3
Constant region amino acid sequences of human IgG heavy chains and human kappa light chain

| | |
|---|---|
| HC | IgG4 (S228P) mutant constant region amino acid sequence (EU Numbering) |
| | ASTKGPSVFP LAPCSRSTSE STAALGCLVK DYFPEPVTVS WNSGALTSGV HTFPAVLQSS |
| | GLYSLSSVVT VPSSSLGTKT YTCNVDHKPS NTKVDKRVES KYGPPCPPCP APEFLGGPSV |
| | FLFPPKPKDT LMISRTPEVT CVVVDVSQED PEVQFNWYVD GVEVHNAKTK PREEQFNSTY |
| | RVVSVLTVLH QDWLNGKEYK CKVSNKGLPS SIEKTISKAK GQPREPQVYT LPPSQEEMTK |
| | NQVSLTCLVK GFYPSDIAVE WESNGQPENN YKTTPPVLDS DGSFFLYSRL TVDKSRWQEG |
| | NVFSCSVMHE ALHNHYTQKS LSLSLGK (SEQ ID NO: 212) |
| LC | Human kappa constant region amino acid sequence |
| | RTVAAPSVFI FPPSDEQLKS GTASVVCLLN NFYPREAKVQ WKVDNALQSG NSQESVTEQD |
| | SKDSTYSLSS TLTLSKADYE KHKVYACEVT HQGLSSPVTK |
| | SFNRGEC (SEQ ID NO: 213) |
| HC | IgG4 (S228P) mutant constant region amino acid sequence lacing C-terminal lysine (K)(EU |
| | Numbering) |
| | ASTKGPSVFP LAPCSRSTSE STAALGCLVK DYFPEPVTVS WNSGALTSGV HTFPAVLQSS |
| | GLYSLSSVVT VPSSSLGTKT YTCNVDHKPS NTKVDKRVES KYGPPCPPCP APEFLGGPSV |
| | FLFPPKPKDT LMISRTPEVT CVVVDVSQED PEVQFNWYVD GVEVHNAKTK PREEQFNSTY |
| | RVVSVLTVLH QDWLNGKEYK CKVSNKGLPS SIEKTISKAK GQPREPQVYT LPPSQEEMTK |
| | NQVSLTCLVK GFYPSDIAVE WESNGQPENN YKTTPPVLDS DGSFFLYSRL TVDKSRWQEG |
| | NVFSCSVMHE ALHNHYTQKS LSLSLG (SEQ ID NO: 214) |
| HC | IgG1 wild type |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVDVS HEDPEVKFNW YVDGVEVHNA KTKPREEQYN |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 215) |
| HC | IgG1 (N297A) mutant constant region amino acid sequence (EU Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVDVS HEDPEVKFNW YVDGVEVHNA KTKPREEQYA |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 216) |
| HC | IgG1 (D265A, P329A) mutant constant region amino acid sequence (EU Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVAVS HEDPEVKFNW YVDGVEVHNA KTKPREEQYN |
| | STYRVVSVLT VLHQDWLNGK EYKCKVSNKA LAAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 217) |
| HC | IgG1 (L234A, L235A) mutant constant region amino acid sequence (EU Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPEAAGG |
| | PSVFLFPPKP KDTLMISRTP EVTCVVVDVS HEDPEVKFNW YVDGVEVHNA KTKPREEQYN |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 218) |

TABLE 4
Amino acid sequences of the heavy and light chain
leader sequences for humanized mAbs BAP049-Clone-
B and BAP049-Clone-E

| | | |
|---|---|---|
| BAP049- | HC | MAWVWTLPFLMAAAQSVQA (SEQ ID NO: 221) |
| Clone-B | LC | MSVLTQVLALLLLWLTGTRC (SEQ ID NO: 222) |
| BAP049- | HC | MAWVWTLPFLMAAAQSVQA (SEQ ID NO: 221) |
| Clone-E | LC | MSVLTQVLALLLLWLTGTRC (SEQ ID NO: 222) |

### Other Exemplary Anti-PD-1 Antibodies for Use in the Combinations Described Herein

In one embodiment, the anti-PD-1 antibody is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody comprises one or more of the CDR sequences (or collectively all of the CDR sequences), the heavy chain or light chain variable region sequence, or the heavy chain or light chain sequence of Nivolumab, e.g., as disclosed in Table 5.

In one embodiment, the anti-PD-1 antibody is Pembrolizumab (Merck & Co), also known as Lambrolizumab, MK-3475, MK03475. SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody comprises one or more of the CDR sequences (or collectively all of the CDR sequences), the heavy chain or light chain variable region sequence, or the heavy chain or light chain sequence of Pembrolizumab, e.g., as disclosed in Table 5.

In one embodiment, the anti-PD-1 antibody is Pidilizumab (CureTech), also known as CT-011. Pidilizumab and other anti-PD-1 antibodies are disclosed in Rosenblatt, J. et al. (2011) J immunotherapy 34(5): 409-18, U.S. Pat. Nos. 7,695,715, 7,332,582, and 8,686,119, incorporated by reference in their entirety. In one embodiment, the anti-PD-1 antibody comprises one or more of the CDR sequences (or collectively all of the CDR sequences), the heavy chain or light chain variable region sequence, or the heavy chain or light chain sequence of Pidilizumab, e.g., as disclosed in Table 5.

In one embodiment, the anti-PD-1 antibody is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody comprises one or more of the CDR sequences (or collectively all of the CDR sequences), the heavy chain or light chain variable region sequence, or the heavy chain or light chain sequence of MEDI0680.

In one embodiment, the anti-PD-1 antibody is REGN2810 (Regeneron). In one embodiment, the anti-PD-1 antibody comprises one or more of the CDR sequences (or collectively all of the CDR sequences), the heavy chain or light chain variable region sequence, or the heavy chain or light chain sequence of REGN2810.

In one embodiment, the anti-PD-1 antibody is PF-06801591 (Pfizer). In one embodiment, the anti-PD-1 antibody comprises one or more of the CDR sequences (or collectively all of the CDR sequences), the heavy chain or light chain variable region sequence, or the heavy chain or light chain sequence of PF-06801591.

In one embodiment, the anti-PD-1 antibody is BGB-A317 or BGB-108 (Beigene). In one embodiment, the anti-PD-1 antibody comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody is INCSHR1210 (Incyte), also known as INCSHR01210 or SHR-1210. In one embodiment, the anti-PD-1 antibody comprises one or more of the CDR sequences (or collectively all of the CDR sequences), the heavy chain or light chain variable region sequence, or the heavy chain or light chain sequence of INCSHR1210.

In one embodiment, the anti-PD-1 antibody is TSR-042 (Tesaro), also known as ANB011. In one embodiment, the anti-PD-1 antibody comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

TABLE 5
Amino acid sequences of other exemplary anti-PD-1
antibodies
Nivolumab

| | | |
|---|---|---|
| SEQ ID NO: 36 | HC | QVQLVESGGGVVQPGRSLRLDCKASGITFS |
| | | NSGMHWRQAPGKGLEWVAVIWYDGSKR |
| | | YYADSVKGRFTISRDNSKNTLFLQMNSLRA |
| | | EDTAVYYCATNDDYWGQGTLVTVSSASTK |
| | | GPSVFPLAPCSRSTSESTAALGCLVKDYFP |
| | | EPVTVSWNSGALTSGVHTFPAVLQSSGLYS |
| | | LSSVVTVPSSSLGTKTYTCNVDHKPSNTKV |
| | | DKRVESKYGPPCPPCPAPEFLGGPSVFLFP |
| | | PKPKDTLMISRTPEVTCVVVDVSQEDPEVQ |
| | | FNWYVDGVEVHNAKTKPREEQFNSTYRVV |
| | | SVLTVLHQDWLNGKEYKCKVSNKGLPSSIE |
| | | KTISKAKGQPREPQVYTLPPSQEEMTKNQV |
| | | SLTCLVKGFYPSDIAVEWESNGQPENNYKT |
| | | TPPVLDSDGSFFLYSRLTVDKSRWQEGNVF |
| | | SCSVMHEALHNHYTQKSLSLSLGK |
| SEQ ID NO: 37 | LC | EIVLTQSPATLSLSPGERATLSCRASQSVSS |
| | | YLAWYQQKPGQAPRLLIYDASNRATGIPAR |
| | | FSGSGSGTDFTLTISSLEPEDFAVYYCQOS |
| | | SNWPRTFGQGTKVEIKRTVAAPSVFIFPPS |
| | | DEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |

Pembrolizumab

| | | |
|---|---|---|
| SEQ ID NO: 225 | HC | QVQLVQSGVEVKKPGASVKVSCKASGYTF |
| | | TNYYMYWVRQAPGQGLEWMGGINPSNGG |
| | | TNFNEKFKNRVTLTTDSSTTTAYMELKSLQF |
| | | DDTAVYYCARRDYRFDMGFDYWGQGTTV |
| | | TVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVD |
| | | HKPSNTKVDKRVESKYGPPCPPCPAPEFLG |
| | | GPSVFLFPPKPKDTLMISRTPEVTCVVVDVS |
| | | QEDPEVQFNWYVDGVEVHNAKTKPREEQF |
| | | NSTYRVVSVLTVLHQDWLNGKEYKCKVSN |
| | | KGLPSSIEKTISKAKGQPREPQVYTLPPSQE |
| | | EMTKNQVSLTCLVKGFYPSDIAVEWESNGQ |
| | | PENNYKTTPPVLDSDGSFFLYSRLTVDKSR |
| | | WQEGNVFSCSVMHEALHNHYTQKSLSLSL |
| | | GK |
| SEQ ID NO: 39 | LC | EIVLTQSPATLSLSPGERATLSCRASKGVST |
| | | SGYSYLHWYQQKPGQAPRLLIYLASYLESG |
| | | VPARFSGSGSGTDFTLTISSLEPEDFAVYYC |
| | | QHSRDLPLTFGGGTKVEIKRTVAAPSVFIFP |
| | | PSDEQLKSGTASVVCLLNNFYPREAKVQW |
| | | KVDNALQSGNSQESVTEQDSKDSTYSLSST |
| | | LTLSKADYEKHKVYACEVTHQGLSSPVTKS |
| | | FNRGEC |

Pidilizumab

| | | |
|---|---|---|
| SEQ ID NO: 40 | HC | QVQLVQSGSELKKPGASVKISCKASGYTFT |
| | | NYGMNWVRQAPGQGLQWMGWINTDSGE |
| | | STYAEEFKGRFVFSLDTSVNTAYLQITSLTA |
| | | EDTGMYFCVRVGYDALDYWGQGTLVTVSS |
| | | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPS |
| | | NTKVDKRVEPKSCDKTHTCPPCPAPELLGG |
| | | PSVFLFPPKPKDTLMISRTPEVTCVVVDVSH |
| | | EDPEVKFNWYVDGVEVHNAKTKPREEQYN |
| | | STYRVVSVLTVLHQDWLNGKEYKCKVSNK |
| | | ALPAPIEKTISKAKGQPREPQVYTLPPSREE |
| | | MTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPG |
| | | K |
| SEQ ID NO: 41 | | EIVLTQSPSSLSASVGDRVTITCSARSSVSY |
| | | MHWFQQKPGKAPKLWIYRTSNLASGVPSR |
| | | FSGSGSGTSYCLTINSLQPEDFATYYCQQR |
| | | SSFPLTFGGGTKLEIKRTVAAPSVFIFPPSD |
| | | EQLKSGTASVVCLLNNFYPREAKVQWKVD |
| | | NALQSGNSQESVTEQDSKDSTYSLSSTLTL |
| | | SKADYEKHKVYACEVTHQGLSSPVTKSFNR |
| | | GEC |

Combination Treatment of Neurodegenerative Diseases, Such as ALS, with BLZ945

To date there are currently two approved drugs, riluzole and edaravone, for the treatment of ALS. The primary hepatic metabolic pathways of riluzole biotransformation in humans may involve direct glucuronidation of riluzole (involving the glucurotransferase isoform UGT-HP4) and oxidation of riluzole to N-hydroxyriluzole by CYP1A2 and CYP1A1, followed by rapid glucuronidation (Sanderink et al 1997). Furthermore, riluzole was shown to be a substrate for breast cancer resistance protein (BCRP) and P-glycoprotein (P-gp) (Milane et al 2009). The pharmacokinetics of BLZ945 are not anticipated to be affected by co-administration of riluzole.

Therefore in one embodiment riluzole is administered in combination with the BLZ945 dosing regimens disclosed herein, for the treatment of neurodegenerative diseases such as ALS multiple sclerosis (MS) or Alzheimers disease.

No significant PK interaction is expected when edaravone is co-administered with BLZ945. Edaravone is an intravenous therapy. Glucuronide conjugation is the primary pathway for edaravone metabolism and eight UGTs (UGT1A1, UGT1A6, UGT1A7, UGT1A8, UGT1A9, UGT1A10, UGT2B7, and UGT2B17) were found to contribute to the production of a significant amount of glucuronide metabolites (Dash et al. 2018). The findings from in vitro studies demonstrated that, at a clinical dose, edaravone and its metabolites are not expected to potentially inhibit CYP enzymes, UGTs and transporters in humans. The pharmacokinetics of edaravone are not expected to be significantly affected by inhibitors of CYP enzymes, UGTs, or major transporters.

Therefore, in one embodiment edaravone is administered in combination with the BLZ945 dosing regimens disclosed herein, for the treatment of neurodegenerative diseases such as ALS, multiple sclerosis or Alzheimer's disease.

### EXAMPLES

### Example 1: A Phase I/II, Open-Label, Multi-Center Study of the Safety and Efficacy of BLZ945 as Single Agent and in Combination with Spartalizumab in Adult Patients with Advanced Solid Tumors

The purpose of this first-in-human (FIH) study of BLZ945 given as a single agent or in combination with spartalizumab is to characterize the safety, tolerability, pharmacokinetics (PK), pharmacodynamics, and anti-tumor activity of BLZ945, administered orally, as a single agent or in combination with spartalizumab, administered intravenously (i.v.) in adult patients with advanced solid tumors.

### Study Design:

This study is a FIH, open-label, multi-center, phase I/II, study which consists of a phase I dose escalation part of BLZ945 as single agent (including a separate Japanese BLZ945 single agent dose escalation arm) and in combination with spartalizumab. Alternative dosing regimens of BLZ945 may be evaluated. BLZ945 is being administered orally, and spartalizumab is being administered i.v. every four weeks until patient experiences unacceptable toxicity, progressive disease and/or treatment is discontinued at the discretion of the Investigator or the patient.

### Inclusion Criteria (Dose Escalation Part in Advanced Solid Tumors):

Phase I: Patients with advanced/metastatic solid tumors (including lymphoma), with measurable or unmeasurable disease as determined by Response Evaluation Criteria in Solid Tumors (RECIST) version 1.1 or RANO (glioblastoma) who have progressed despite standard therapy or are intolerant of standard therapy, or for whom no standard therapy exists.
Patients must have a site of disease amenable to biopsy, and be a candidate for tumor biopsy according to the treating institution's guidelines. Patient must be willing to undergo a new tumor biopsy at screening, and during treatment. Exceptions may be considered for glioblastoma patients after documented discussion with Novartis.
Phase II: Patients with advanced/metastatic tumors in the below selected indications, with at least one measurable lesion as determined by RECIST v1.1 or Response Assessment in Neuro-Oncology Criteria RANO (glioblastoma) or the Guidelines for efficacy evaluation in Hodgkin and non-Hodgkin lymphoma studies for lymphoma indication(s)
   advanced pancreatic cancer that failed to respond or progressed on or after treatment with standard of care
   advanced triple negative breast cancer, that failed to respond to standard treatment or progressed on or after standard treatment
   recurrent glioblastoma that failed to respond or progressed on radiotherapy and temozolomide except for patients with O6-methylguanine-DNA methyltransferase (MGMT) unmethylated newly diagnosed gliobastoma who may have received radiation therapy only.

### Dosing Regimen:

BLZ945 dosing is being evaluated on the following schedules, 7 days on/7 days off (i.e., administer BLZ945 every day for 7 days and suspend for 7 days), once weekly (Q1W), and 4 days on/10 days off (i.e., administer BLZ945 every day for 4 days and suspend for 10 days). For each of these schedules, once per day (OD) or twice per day (BID) dosing may be evaluated. For once daily administration, patients should take their dose at approximately the same time in the morning. For the twice a day dosing, the first dose should be taken in the morning and the second dose should be taken approximately 10 to 12 hours after the morning dose.

On days that PK samples are obtained, the patient should take BLZ945 during the clinic visit after the pre-dose PK samples and prior to post-dose PK samples, when instructed by the study staff. Patients should take BLZ945 on an empty stomach (i.e. fast from food and drink, except water) at least 1 hour before or 2 hours after a meal. Each dose should be taken with a glass of water. Patients should be instructed to swallow whole capsules and not to chew or open them.

TABLE 6
Dose and Treatment Schedule

| | Pharmaceutical | | |
|---|---|---|---|
| Study | form and route of | | Frequency and/or |
| Treatment | administration | Dose | regimen |
| BLZ945 | Capsule, | 150 mg | Once Daily or twice |
| | per os (p.o.) | (starting | a day either: |
| | | dose) | 7 days on/7 days |
| | | | off, or 4 days |
| | | | on/10 days off |
| | | | or QIW (once a week) |
| spartalizumab | Powder for | 400 mg | Every 4 weeks |
| | solution for | | |
| | i.v. infusion | | |

All patients treated with either BLZ945 single agent or in combination with spartalizumab will begin study treatment on Cycle 1 Day 1. Each cycle will consist of 28 days.

### Starting Dose of BZL945 as a Single Agent (SA)

In order to evaluate the safety, PK and antitumor activity of BLZ945 across a range of doses, the recommended starting dose in this study will be 150 mg on a 7d on/7 off schedule. Once per week dosing (Q1W or 4d on/10d off dosing may also be explored in parallel if deemed appropriate based on emerging PK and safety assessments. The dose escalation decision making will be guided by a Bayesian logistic regression model (BLRM) with Escalation With Overdose Control (EWOC) principle based on DLT data in the context of available safety, PK and PD information. This open-label dose escalation study design using a BLRM is a well-established method to estimate the MTD/RP2D in cancer patients. The adaptive BLRM will be guided by the EWOC principle to control the risk of DLT in future patients on study. The use of Bayesian response adaptive models for small datasets has been accepted by European Medicines Agency (Guideline on clinical trials in small populations, 13 Feb. 2007) and endorsed by numerous publications (Zacks and Hersh 1998, Neuenschwander et al 2008, Neuenschwander et al 2010), and its development and appropriate use is one aspect of the FDA's Critical Path Initiative. The starting dose of new schedule(s) will be equal or lower than the maximum dose that have been previously tested and met the Escalation with Overdose Control (EWOC) criteria for the Bayesian Logistic Regression Model (BLRM) on the 7d on/7d off schedule.

The Japanese dose escalation for BLZ945 single agent will run separately from the ongoing non-Japanese dose escalation with a starting dose deemed appropriate based on emerging PK and safety assessments and meeting the EWOC criteria of both the BLRM in the global dose escalation arm and that for the Japan specific escalation.

Twice daily dosing schedules may also be explored if deemed appropriate. The cumulative starting dose (i.e., morning dose plus evening dose) will not be higher than the dose of the once daily administration that has been tested and shown to satisfy the EWOC criteria using the Bayesian Hierarchical Logistic Regression Model (BHLRM) following a discussion with participating Investigators during a dose escalation teleconference.

### Starting Dose of BZL945 and Spartalizumab Combination

In order to administer a safe dose of BLZ945 in the combination with spartalizumab, the starting dose of BLZ945 will meet the following:
Dose has already been tested in the single agent escalation arm
At least one dose level below the highest investigated single agent dose for BLZ945 that satisfied the EWOC criterion under the appropriate single agent BLRM, is deemed appropriate based on emerging PK and safety assessments, and meets the EWOC criteria based on the BLRM of the combination model.
Not less than the single agent starting dose (i.e. 150 mg) unless DLT are observed at the starting dose.

In both combination dose escalations, spartalizumab will be administered at a fixed dose of 400 mg i.v. every 4 weeks, which has been shown to be well tolerated.

TABLE 7
Provisional Dose Levels (BLZ945 single agent)

| | Proposed dose | Increment from |
|---|---|---|
| Dose Level | of BLZ945* | previous dose |
| −1** | 100 mg | −33% |
| 1 | 150 mg | Starting dose |
| 2 | 300 mg | 100% |
| 3 | 600 mg | 100% |
| 4 | 900 mg | 50% |

*It is possible for additional and/or intermediate dose levels to be added during the course of the study Cohorts may be added at any dose level below the MTD in order to better understand safety, PK or PD.
**Dose level −1 represents treatment doses for patients requiring a dose reduction from the starting dose level. No dose reduction below dose level −1 is permitted for this study.

TABLE 8
Provisional Dose Levels (BLZ945 in
combination with spartalizumab)

| | Proposed dose | increment from | Proposed dose of |
|---|---|---|---|
| Dose Level | of BLZ945* | previous dose | spartalizumab |
| −1** | 100 mg | −33% | 400 mg |
| 1 | 150 mg | Starting dose | 400 mg |
| 2 | 300 mg | 100% | 400 mg |
| 3 | 600 mg | 100% | 400 mg |
| 4 | 900 mq | 50% | 400 mg |

*It is possible for additional and/or intermediate dose levels to be added during the course of the study Cohorts may be added at any dose level below the MTD in order to better understand safety, PK or PD.
**Dose level −1 represents treatment doses for patients requiring a dose reduction from the starting dose level. No dose reduction below dose level −1 is permitted for this study.

### Treatment Duration

A patient may continue treatment with BLZ945 single agent until the patient experiences unacceptable toxicity, confirmed disease progression per irRC (or per iRANO for glioblastoma patients) or progressive (metabolic) disease per the Guidelines for efficacy evaluation in Hodgkin and non-Hodgkin lymphoma studies for r/r lymphoma patients and/or treatment is discontinued at the discretion of the investigator or the patient.

A patient may continue treatment with BLZ945 in combination with spartalizumab until the patient experiences unacceptable toxicity, confirmed disease progression per irRC (or per iRANO for glioblastoma patients) or progressive (metabolic) disease per the Guidelines for efficacy evaluation in Hodgkin and non-Hodgkin lymphoma studies for r/r lymphoma patients and/or treatment is discontinued at the discretion of the investigator or the patient. In the first 24 weeks of treatment, patients will not be withdrawn from the study due to progressive disease per RECIST v1.1 (or per RANO for glioblastoma patients or per the Guidelines for efficacy evaluation in Hodgkin and non-Hodgkin lymphoma studies for r/r lymphoma patients).

### Example 2: Modeling and Simulation of Impact of Holiday Dosing on ALT AE's

During the course of the clinical study in Example 1, adverse events (AEs) of all grades and regardless of relationship to study treatment were reported in all 68 patients (100%) treated with single agent BLZ945 and in all 46 patients (100%) treated with the combination regimen. The most frequently reported AEs (>10%) suspected to be related to BLZ945 single agent were aspartate aminotransferase (AST) increased, nausea, vomiting, alanine aminotransferase (ALT) increased, fatigue, amylase increased, blood creatine phosphokinase increased and decreased appetite. The most frequently reported suspected AEs (>10%) with the combination treatment were AST increased, ALT increased, pruritus, fatigue, nausea, rash and vomiting. Three suspected SAEs were reported in the single agent arm. These SAEs were Grade 3 AST increased, Grade 3 asthenia and Grade 4 sudden death. In the combination arm, 7 SAEs in 4 patients were reported as suspected to be related to study treatment, including Grade 3 AST increased, Grade 4 ALT increased, Grade 4 immune mediated hepatitis, Grade 3 colitis, and Grade 2 stomatitis with Grade 1 pyrexia and Grade 2 rash maculopapular.

The preliminary pharmacokinetics (PK) of single dose and multiple doses of oral BLZ945 when administered alone and in combination with spartalizumab has been assessed in the ongoing study CBLZ945X2101. BLZ945 showed rapid absorption across all tested doses as single agent as well as in combination with spartalizumab. Mean terminal elimination half-life (T½) for BLZ945 ranged from 16.4 to 26.7 hours, and was consistent when given as single dose or multiple doses, across all doses and dosing regimens, and when given alone or in combination with spartalizumab. The analysis of dose normalized Cmax and AUC0-24 hr indicated that the exposure of BLZ945 is less than proportional to dose starting from 600 mg dose, when given alone or in combination with spartalizumab. The analysis of accumulation index (Racc) indicated that once a day dosing for 7 days or 4 days caused more accumulation at the end of the dosing period (mean Racc ranged from 1.54 to 2.20) than Q1 W regimen (mean Racc ranged from 1.07 to 1.24). Based on preliminary data generated to date, spartalizumab does not appear to impact the PK of BLZ945.

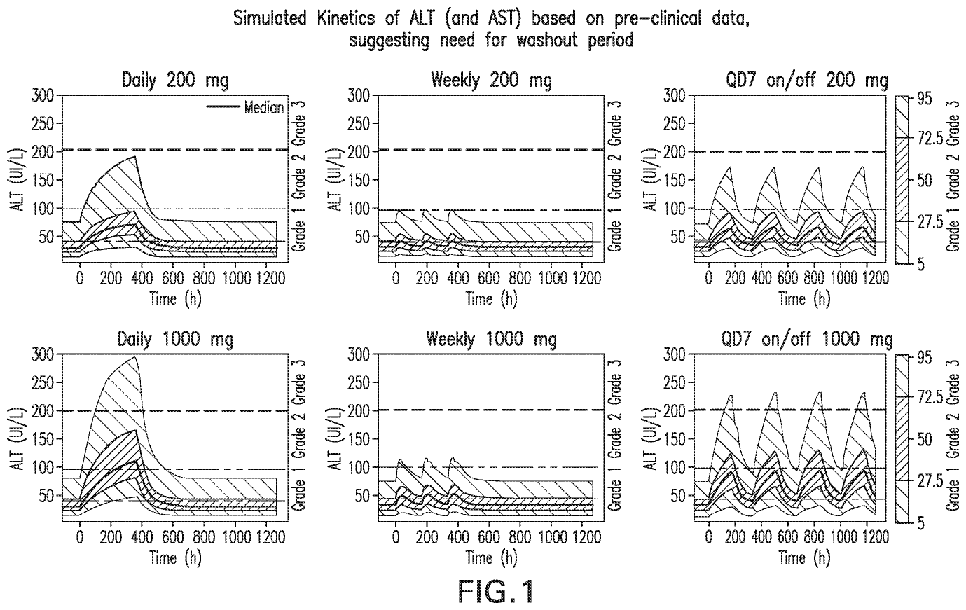

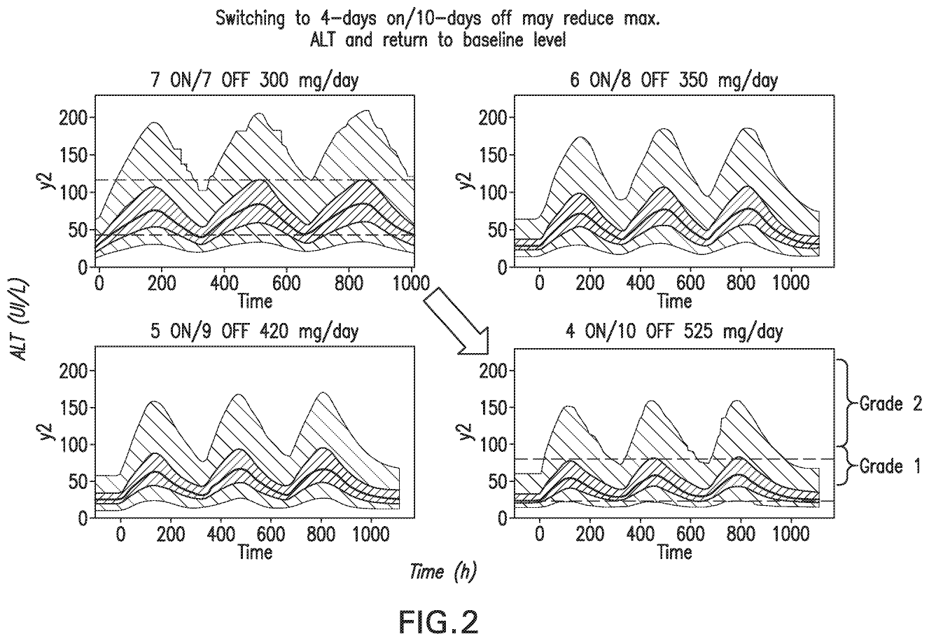

A cynomolgus monkey ALT modeling based on pre-clinical data predicted that dose holidays might reduce the probability of increased ALT (See FIGS. 1 & 2). In addition, data available from the clinical study of Example 1 from 5 patients dosed at 150 mg, and 5 at 300-mg, was used to construct a population PK-ALT model that relates BLZ945 drug concentration to ALT. Simulations were performed using the model in order to predict the probability of grade 1, 2, 3, and 4 ALT AE's. Three different regimens were simulated: 7 days on/7 days off with a 300-mg dose (7 days×300 mg=2100 mg total BLZ945 dosed) and 2100 mg over a period of 14 days Q1W (once weekly), and 4 days on/10 days off. The comparison of these three regiments led to the conclusion that there was a need for a washout period (drug holiday) in order to allow baseline ALT normal value recovery and that there was a decreased probability of such AE's on the 4 days on/10 days off regimen.

This need for a washout period is due to the PK associated with BLZ945, and not the specific disease treated. In fact, macrophages expressing CSF-1R, especially Kupffer cells in the liver, play a role in the clearance of enzymes from circulation, including alanine aminotransferase (ALT), aspartate aminotransferase (AST), and creatine kinase (CK). Inhibition of CSF-1R induces increase in AST and ALT levels, due to depletion of CSF1 R+Kupffer cells (class effect of CSF-1R targeting compounds). Therefore, this dosing on/dosing off dosing regimen (such as dosing cycles of 4 days/10 days off, or of 7 days on/7 days off, etc.) applies generally to the treatment of diseases with BLZ945; for example for the treatment of cancer or neurodegenerative diseases such as ALS or MS, with BLZ945.

### Example 3: MC38 Syngeneic Mouse Model in C57BL16N Mice

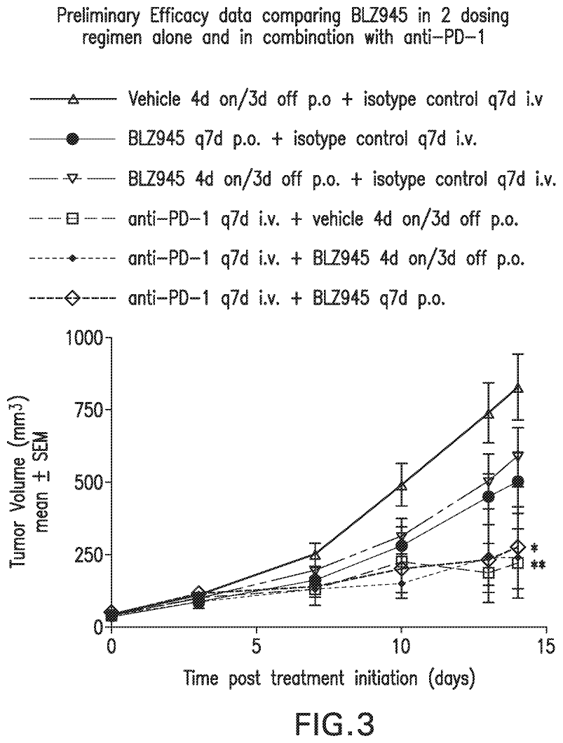

In another set of experiments using the MC38 syngeneic mouse model in C57BL/6N mice two different schedules of BLZ945 were compared for efficacy (FIG. 3) using once weekly dosing of BLZ945 and 4 days on/3 days off dosing of BLZ945 alone and in combination with anti-PD-1. Anti-PD-1 treatment resulted in complete response (CR) in 6/10 animals weekly administration of BLZ945 in combination with anti-PD-1 resulted in 5/10 CRs and a significant tumor growth inhibition when compared to vehicle control animals, corresponding to a % T/C vs. control of 37% (p=3.90×10−2) on day 10 and 32% (p=1.57×10−2) on day 14. Administration of BLZ945 in a 4 days on/3 days off schedule in combination with anti-PD-1 resulted in 4/10 CRs and in a significant tumor growth inhibition on day 10, corresponding to a % T/C vs. control of 28% (p=4.76×10-2). On day 14 post treatment start, there was a trend of tumor growth inhibition, corresponding to a % T/C vs. control of 28%.

Doses of 200 mg/kg BLZ945 and 10 mg/kg anti-PD-1 were given on the schedules indicated and tumor volumes measured on day 14 after study initiation. Statistical significance was calculated using one-way ANOVA with post hoc Dunnett's test for comparison of treatment versus control group.

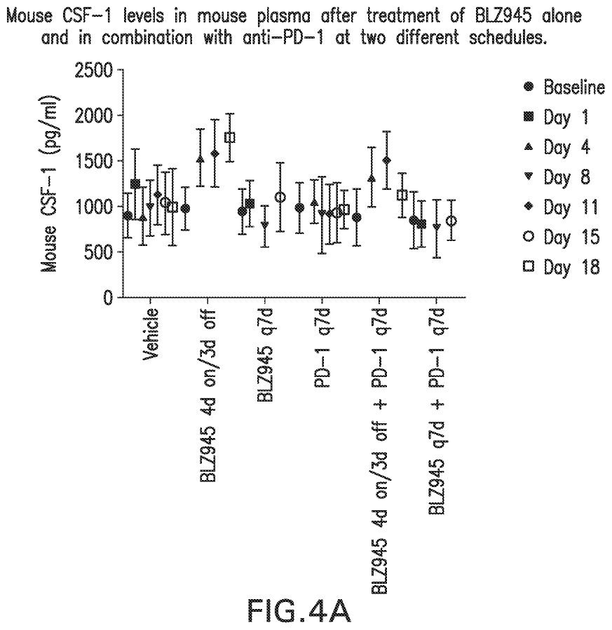

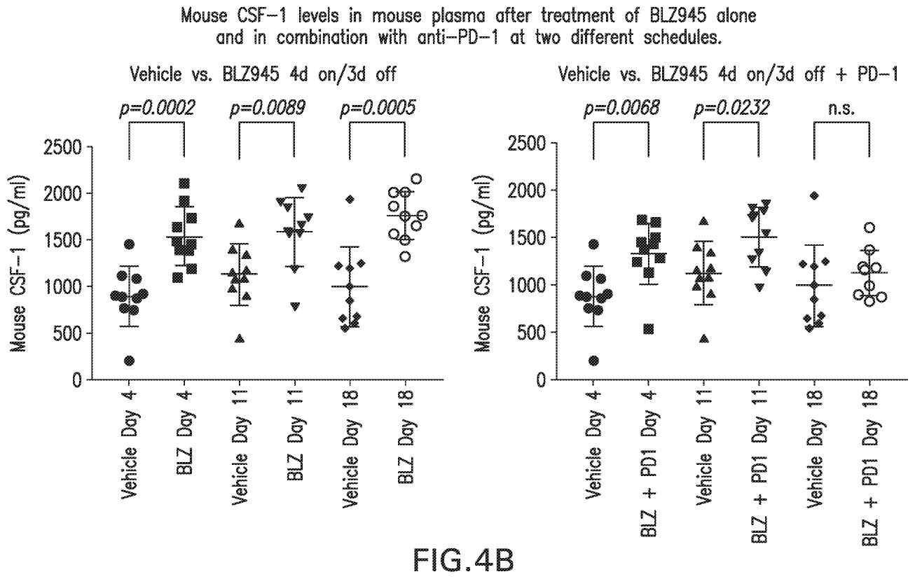

In order to measure increase of CSF1 in the plasma as a biomarker for CSF-1R inhibition blood sampling was performed at baseline and 6 hours later on indicated days (FIG. 4). Only the BLZ945 4 days on/3 days off schedule alone and in combination resulted in a statistically significant increase in CSF-1 plasma levels (FIG. 4).

Mouse CSF-1 levels in plasma. A. Changes in CSF-1 levels over time in the different treatment groups. B. Statistical analysis of CSF-1 levels in vehicle vs. BLZ945 4 days on/3 days off and in vehicle vs. BLZ945 4 days on/3 days off in combination with anti-PD-1. Statistical significance was calculated using a two-tailed, unpaired, non-parametric Mann-Whitney Test was performed to compare treatment to control groups.

In order to access the dynamics of the tumor associated immune cells a repeat study using only the 4 days on/3 days off schedule as a single agent and in combination with anti-PD-1 was performed. A significant decrease in TAM was observed after 10 and 14 days of treatment as well as a transient decrease in Tregs on day 10 after treatment start.

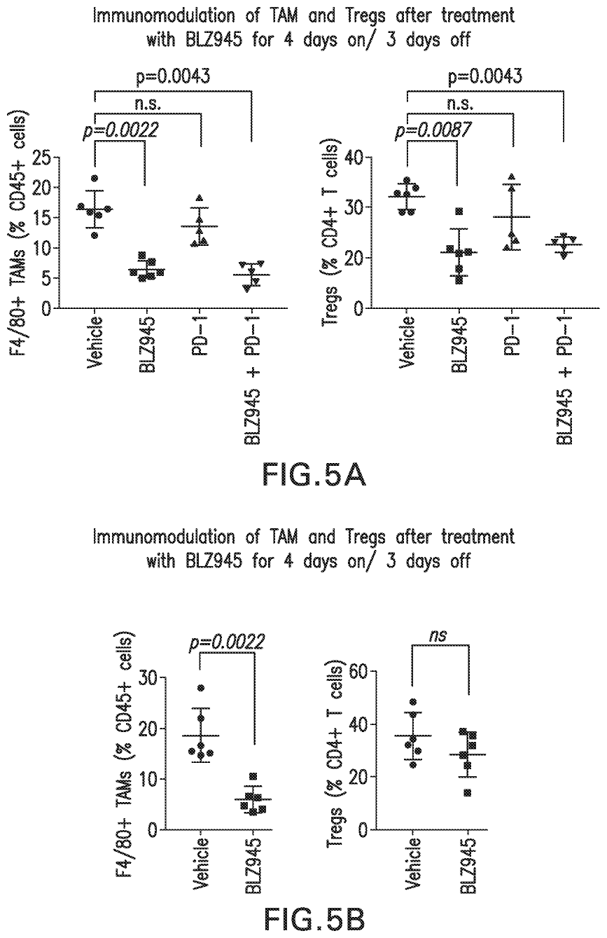

FIG. 5 Analysis of tumor infiltrating macrophages (FIG. 5A) and Tregs (FIG. 5B) upon treatment with BLZ945 in a 4 days on/3 days off schedule on day 10 (A) and day 14 (B) and in combination with anti-PD-1 by flow cytometry. Statistical significance was calculated using a two-tailed, unpaired, non-parametric Mann-Whitney Test was performed to compare treatment to control groups.

Efficacy testing was repeated with a suboptimal dose of anti-PD-1 (5 mg/kg) in order to test for an additive effect of BLZ945 to be observed when given in combination. Interestingly, only the 4 days on/3 days off schedule in combination with anti-PD-1 and not either of the single agent groups dosed with BLZ945 or anti-PD-1 resulted in a statistically significant tumor growth inhibition.

### Example 4: AST/ALT Elevation Across Single Agent Dose Groups for the Compound of Formula (I)

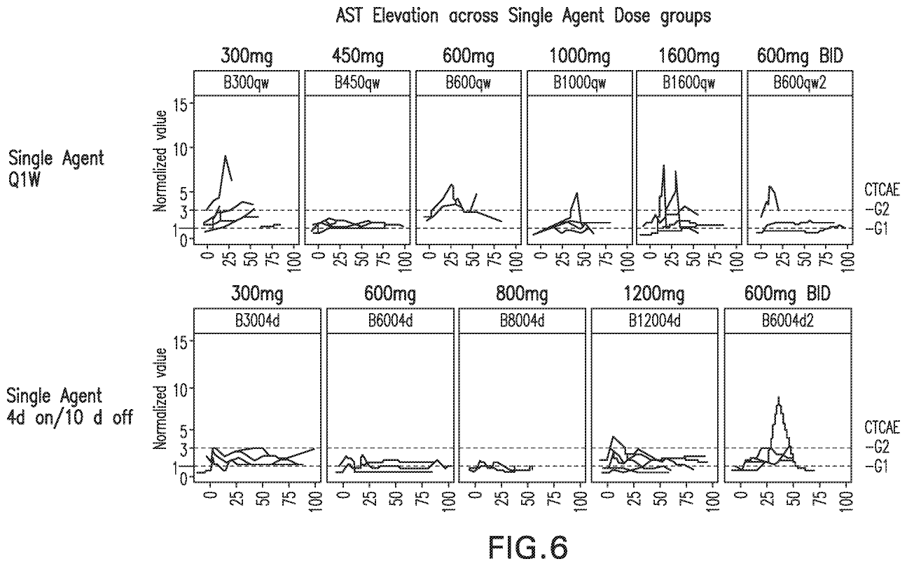

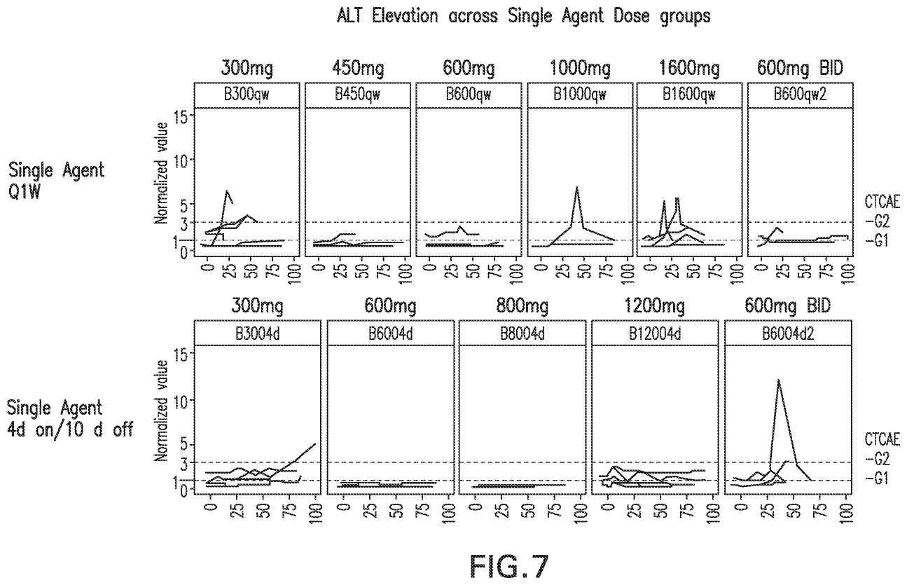

A comparison of AST and ALT elevation across single agent dose groups for the compound of Formula (I) was performed for the once per week (Q1WV) dosing regimen and the 4 days on/10 days off dosing regimen over a range of dosages. FIGS. 6 and 7 show the AST and ALT elevations, respectively. The Common Terminology Criteria for Adverse Events (CTCAE) was used to evaluate AST and ALT levels. Surprisingly, AST and ALT elevations are both better controlled with the 4 days on/10 days off dosing regimen, compared with the Q1W dosing regimen. As seen in FIGS. 6 and 7, the 4 days on/10 days off schedule allows for ALT/AST levels to recover to baseline before next dose, while the Q1 W dose shows spikes above the G2 level for both AST and ALT levels, even at the lowest dose of 300 mg.

### Example 5: PK/PD in ALS Mice Models

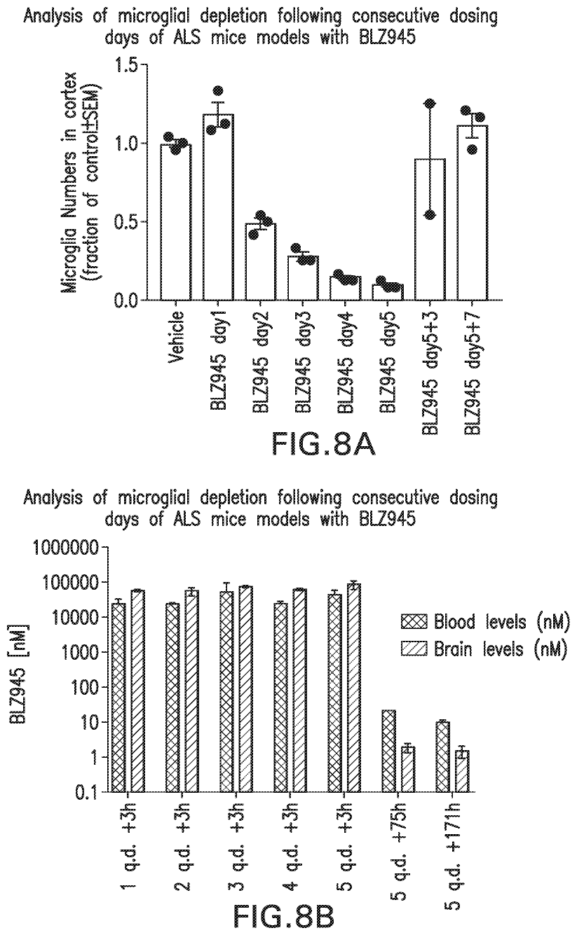

Several studies were performed to understand PK/PD relationships for CNS microglia clearance in mice. In the first study (RD-2019-00100) mice were treated once daily (qd) for between 1 and 5 consecutive days with a dose of 169 mg/kg BLZ945. Additionally, two dose groups received 169 mg/kg BLZ945 for 5 consecutive days followed by either 3 or 7 days of drug withdrawal (washout). Histological analysis indicated that microglia were depleted in the cortex by approximately 50% on day 2 and up to approximately 90% by day 5 (FIG. 8 A). In the washout groups, microglia showed nearly complete brain repopulation (˜80% of vehicle) 3 days following drug removal and complete repopulation by day 7. Pharmacokinetic analysis was performed on blood and brain samples taken from each group at 3 hours after the final dose on the indicated necropsy day. Blood and brain exposures remained constant on consecutive dosing days, indicating lack of accumulation with daily dosing, and with a ratio of approximately 50% brain exposure relative to blood. Exposures dropped by more than 1000-fold following the washout period (FIG. 8 B).

FIG. 8: (A) Microglial depletion in the cortex, as assessed by Iba1/Aif-1 immunohistological staining at necropsy. Sections were analyzed for total number of microglia cell bodies per sample area. Values are group means±SD (n=3). (B) Exposure of BLZ945 in blood and brain 3 hours after the last dose on the day of necropsy. Values are group means (n=3).

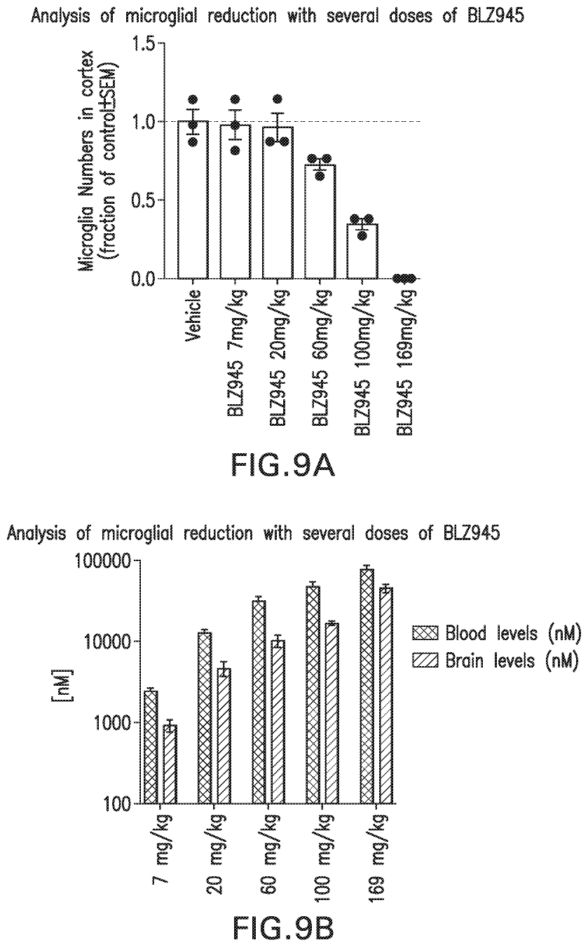

In a follow-up study (RD-2019-00100), mice were treated with BLZ945 for 5 consecutive days with a range of doses from 7 to 169 mg/kg. The two lowest doses of 7 mg/kg and 20 mg/kg were ineffective for microglial depletion. However, a dose-dependent effect was observed with the three higher doses (60, 100, and 169 mg/kg) of BLZ945 that exhibited microglial reductions of 25, 60, and 100%, respectively (FIG. 9 A). Pharmacokinetic analysis indicated linear increases in both blood and brain exposures with increased doses. Here the ratio of brain to blood exposures was approximately 30% at the four lowest doses and approximately 55% at the highest dose (FIG. 9 B).

FIG. 9: Dose-dependent reduction of microglia. (A) Mice were treated for 5 consecutive days with BLZ945 at the indicated doses. Samples were analyzed histologically by Iba1/Aif-1 staining and quantified for total microglia cell bodies per sample area. Data are group means±SD (n=3). (B) Exposure of BLZ945 in blood and brain following 5 consecutive days of dosing at multiple doses. Values are group means±SD (n=3).

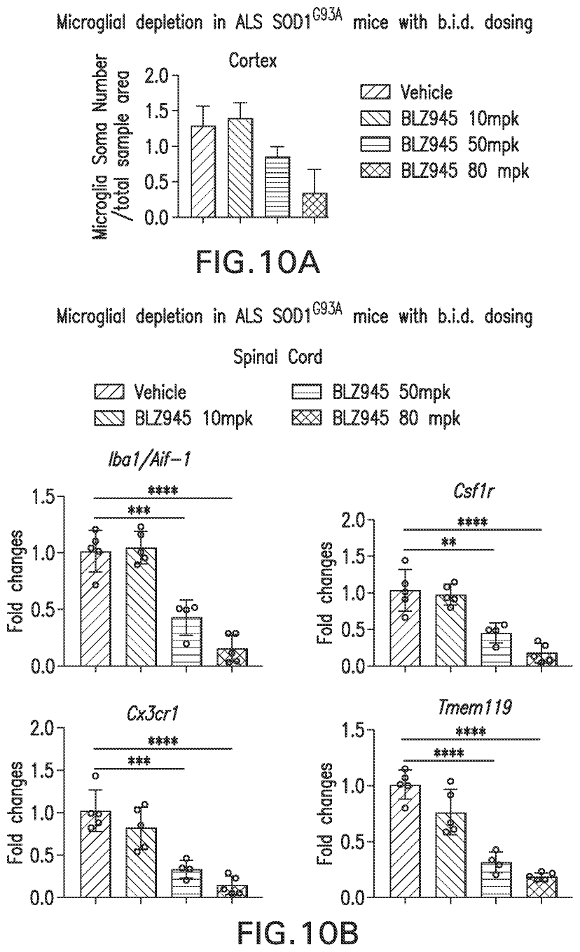

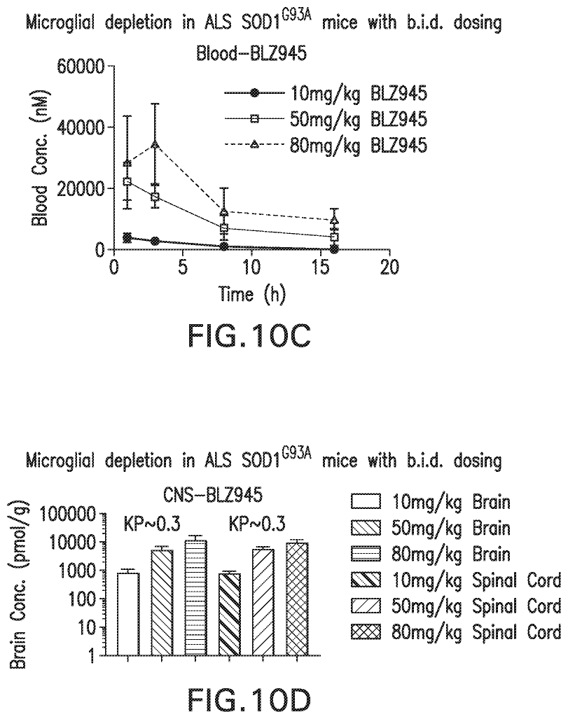

Dosing was examined in the SOD1G93A mouse model of ALS, the most commonly used rodent ALS model (RD-2019-00111). Twice daily dosing (b.i.d.) was examined after 5 consecutive dosing days. Microglia were depleted in the cortex by approximately 25% and 75% following treatment with 50 mg/kg and 80 mg/kg BLZ945. BLZ945 dosed b.i.d. at 10 mg/kg was not effective (FIG. 10 A). Similarly, in spinal cord qPCR analysis of several microglial markers indicated that microglia were depleted with doses of 50 mg/kg and 80 mg/kg BLZ945 by approximately 55% and 85%, respectively (FIG. 10 B). BLZ945 dosed at 10 mg/kg had a small but non-significant effect of reducing some of the markers. Brain and spinal cord analysis showed that drug exposure was about 30% of blood at three hours after the final dose at necropsy (FIG. 10 C/D).

FIG. 10: BLZ945-mediated microglial depletion in the cortex, as assessed by Iba1/Aif-1 immunohistological staining (A) or in spinal cord by qPCR analysis (B) Values are group means±SD (n=3). (C and D) Exposure of BLZ945 in blood and CNS brain and spinal cord 3 hours after the final dose of the study.

### Example 6: Efficacy with BLZ945 in ALS Animal Models

BLZ945 was dosed over an 8-week period in a mouse model of ALS (in house data). BLZ945 was tested for efficacy in preventing or delaying disease progression in the SOD1G93A ALS mouse (RD-2019-00092). This model animal exhibits hind limb weakness and neuromuscular impairment that are detectable beginning between 8 and 10 weeks of age. Normal body weight gain is also impaired in this model. These physiological impairments are considered as mechanistic readouts given that they best recapitulate the clinical disease phenotype (Turner et al 2013). Animals were dosed from 8 to 16 weeks of age with daily (q.d) doses of BLZ945 at either 65 mg/kg, 170 mg/kg, or 170 mg/kg delivered intermittently with a 7 day ON/7 day OFF regimen.

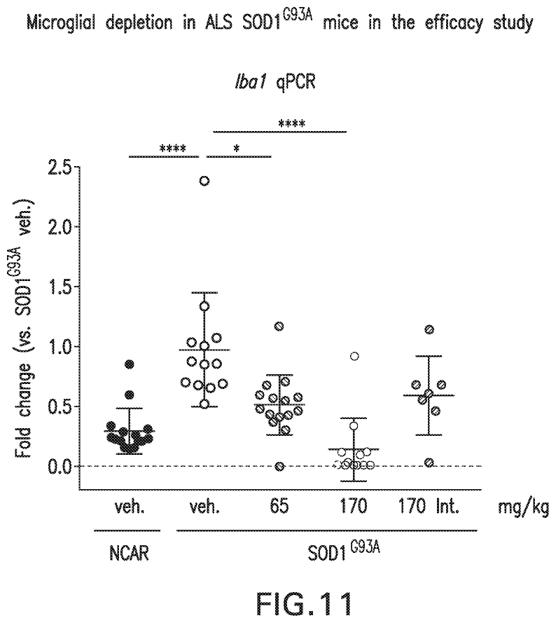

Microglia were depleted by greater than 90% with the high dose (170 mg/kg) of BLZ945 and by about 50% with the low dose (65 mg/kg). The intermittent dosing group was sacrificed at the 7 days off (i.e. 7 days without drug) point of the dosing cycle and exhibited microglia recovery by greater than 50% compared to high dose animals (FIG. 11). Blood and CNS exposure to BLZ945 were measured at 3 hours post-dose on the day of necropsy (Table 8). BLZ945 spinal cord exposure ratios (relative to blood) were consistent across dose groups at approximately 0.4 (Table 9).

TABLE 9
BLZ945 exposure in blood and tissue
across dosing groups (3 h post-dose)

| | | | Spinal |
|---|---|---|---|
| | Blood conc. | Spinal Cord | Cord/Blood |
| Group | (pmol/mL) | Conc.(pmol/g) | ratio |
| BLZ945 65 mg/kg | 29291.6 | 11799.8 | 0.42 |
| BLZ945 170 mg/kg CONT | 66287.8 | 28358.8 | 0.42 |
| BLZ945 170 mg/kg INT | 50427.5 | 18573.5 | 0.36 |

FIG. 11: Microglia depletion in the lumbar spinal cord, assessed by Iba1 transcript expression by qPCR. Samples were collected at necropsy on the final day of dosing of the eight-week study. Values are group means±SD (n=12-15). Data are presented as fold changes in comparison to the SOD1 vehicle-treated group. Statistical analyses were by Kruskal-Wallis test with Dunn's multiple comparison

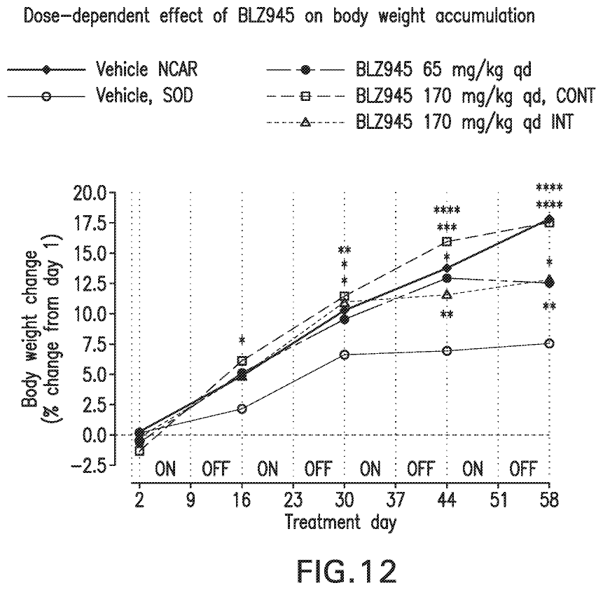

Wild type non-carrier of transgene (NCAR) control animals showed continuous body weight gain (approximately 16%) during the eight-week study period, consistent with normal aging. However, vehicle-treated SOD1G93A control animals exhibited an initial slower rate of gain and eventual plateau by the study mid-point, coincident with disease onset (approximately 7% gain by the end of study). In contrast, SOD1G93A animals treated with BLZ945 continuously at 170 mg/kg showed constant weight gain at the same rate as NCAR mice throughout the study (FIG. 12). The groups treated with low (i.e. 65 mg/kg) and intermittent doses (170 mg/kg 7 days on/7 days off) of BLZ945 exhibited rates similar to NCAR mice early in the study but eventually reaching a plateau in the final weeks of dosing to a level intermediate between the vehicle and high-dose groups (approximately 12%).

FIG. 12: The effect of BLZ945 on body weight change over the eight-week study period. Data are presented as the percentage of body weight change relative to the first dosing day. Values are group means (n=2-15). Statistical analysis was by mixed linear effect model for repeated measures. * p<0.05; ** p<0.01; **** p<0.0001.

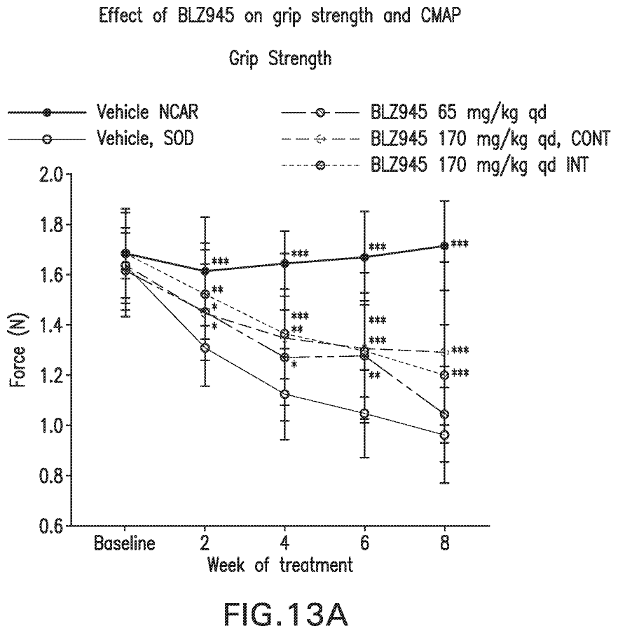

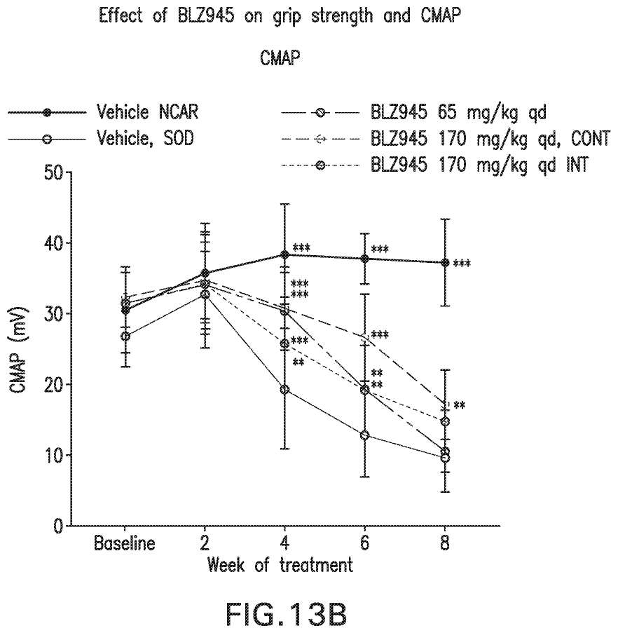

Hind limb grip strength and compound muscle action potentials (CMAP), an assessment of neuromuscular integrity, were measured as mechanistic readouts. In grip strength measurements, SOD1 G93A mice lost muscle force continuously after the 8th week of age. Mice receiving the high dose of BLZ945 (170 mg/kg), either continuously or intermittently, maintained significantly greater grip force throughout the study period. This was intermediate to the vehicle-treated SOD1 G93A and NCAR controls (FIG. 13 A). Animals treated with the low dose of BLZ945 (65 mg/kg) similarly showed significantly higher grip strength, in comparison to vehicle-treated SOD1 G93A controls, up to week 6 of dosing. Further assessment showed that vehicle-treated SOD1G93A controls exhibited a sharp decline in tibialis anterior (TA) muscle innervation, as measured by CMAP, following the 2nd week of the study (FIG. 13 B). However, SOD1 G93A animals treated with BLZ945 in the continuous or intermittent dosing (i.e. 170 mg/kg) groups showed significantly reduced decline in CMAPs throughout the study period. However, CMAPs were still lower than those of NCAR animals from study week 4 onward. SOD1 G93A animals treated with BLZ945 at 65 mg/kg showed significantly higher CMAPs than untreated controls until week 6 of the study.

FIG. 13: (A) The effect of BLZ945 on grip strength over the eight-week study period. Values are group means±SD (n=12-15). Statistical analysis was by mixed linear effect model for repeated measures. * p<0.05; ** p<0.01; *** p<0.001. (B) The effect of BLZ945 on TA muscle CMAP over the eight-week study period. Values are group means±SD (n=12-15). Statistical analysis was by mixed linear effect model for repeated measures. * p<005; ** p<0.01; *** p<0.001.

In summary, the BLZ945 high dose group showed maintenance of normal body weight gain in comparison to vehicle-treated controls, while the low and intermittent dose groups showed intermediate effects. Additionally. BLZ945 showed dose-dependent delay of disease-related impairments in grip strength and muscle innervation in the continuous dosing groups, while the intermittent group showed moderate effects similar to the low dose group. This efficacy was associated at necropsy with dose-dependent depletion of microglia from the spinal cord in the continuous dose groups and partial repopulation of microglia in the intermittent group (analyzed at the seventh day of the off-drug period).

In a preclinical 13-wk study (Study number 1779034), cynomolgus monkey were treated with 30, 60 and 200 mg/kg/day BLZ945 on daily oral dosing or at 60 and 200 mg/kg/day on 7 days-on and 7 days-off treatment cycles. With either regimen, the dosing period was for 91 days followed by a drug-free period up to a total of 14d days. Plasma was collected at pre-dose, during the dosing period at different time points and during the recovery time, and CSF was collected at necropsy at end of dosing and recovery periods. Soluble TREM2, a biomarker of microglial activation, was measured in plasma and CSF.

In plasma, treatment with BLZ945 reduced TREM2 in a dose- and time-dependent manner to 40-45% of the predose value at the mid-continuous and the high continuous dosing (daily dose) regimen. After cessation of dosing, plasma TREM2 returned to baseline within 7 days. In the cyclic dosing (7 days-on 7 days-off cyclic dose) regimen, TREM2 plasma values were decreased at end of on-treatment weeks for both mid- and high-doses, and were in the range of control values at end of off-treatment week.

In CSF, a dose-dependent reduction of soluble TREM2 in monkeys treated with BLZ945 for 3 months in both continuous (up to about 25-fold decrease at high dose) and to a lesser extent in on-off dosing (up to about 4.6-fold decrease at high dose) regimens was observed. This significant reduction of CSF TREM2 in treated monkeys, indicated target engagement by BLZ945 including in the on-off regimen, and reflects its intended pharmacology. At the end of the recovery phase, soluble TREM2 values in the high daily, and the high on/off treatment groups returned to that of untreated controls.

These data indicate target engagement by BLZ945 and show that soluble TREM2 can be used to monitor BLZ945 pharmacology response in monkeys centrally in CSF and in the periphery in plasma.

### Example 7: Predicted Therapeutic Dose

A PK/PD (drug concentration in plasma/microglia in brain) relationship was established in C57b1/6 and SOD-1 mice. The BLZ945 PK in ALS subjects was predicted based on preliminary PK data in study CBLZ945X2101 (assumes similar BLZ945 pharmacokinetics in ALS subjects was to that in oncology subjects). The PK/PD relationship established in mice was then applied to the PK in ALS subjects to predict PD (microglia in brain) in ALS subjects (assuming similar PK/PD relationship in man and mice). The results of the PK/PD modeling indicate that 4 days of treatment with the selected starting dose of 300 mg BLZ945 once daily is predicted to result in a reduction of 10-12% in brain microglia. A 1200 mg qd dose is expected to lead to a 40-78% reduction in brain microglia (Table 10).

TABLE 10
Observed and predicted preliminary BLZ945 PK parameters on Day 4,
safety margins and predicted brain microglia decrease following
once daily oral administration of 150, 300, 600, 800, and 1200 mg

| | PK parameter (Day 4)¹⁾ | | |
|---|---|---|---|
| Daily dose | Cmax | AUC0-24 h | Predicted Brain |
| (mg) | (ng/mL) | (h*ng/mL) | Microglia Decrease |
| 150 | 6950²⁾ | 101500²⁾ | ≤5 |
| 300 | 13900 | 203000 | 10-12 |
| 600 | 24500 | 378000 | 35-50 |
| 800 | 22700 | 413000 | 35-50 |
| 1200 | 32700 | 599000 | 40-78 |

¹⁾Preliminary PK parameters from study CBLZ945X2101
²⁾Estimated PK parameters on Day 4 from PK data in oncology subjects, assuming dose-linear and time-independent PK in the dose range 150-300 mg

### Example 8: Clinical Study on/Off Dosing for the Treatment of ALS

CBLZ945C12201 is a multiple ascending dose study with the starting dose of 300 mg daily for four days treatment and a maximum dose of 1200 mg daily for four days treatment. Patients with ALS will receive daily treatment with BLZ945 for 4 days. Three separate cohorts of daily dosing of: 300 mg/day, 600 mg/day, or 1200 mg/day, respectively, will be used. An additional two cohorts will be used to investigate doses between 300 mg/day and 1200 mg/day.

As mentioned above, nausea, vomiting have been commonly observed to occur with single-agent administration of BLZ945 in the first-in-human trial (CBLZ945X2101). Asymptomatic serum enzyme elevation including AST, ALT, CK and alkaline phosphatase elevations were also observed Asymptomatic serum enzyme increases were also reported in clinical trials with monoclonal antibodies against CSF-1R (FPA008 and emactuzumab), against CSF-1 ligand (MCS110) and CSF-1R inhibitor PLX3397 (Rugo et al 2014, Cassier et al 2015, Pognan et al 2019, Zhou et al 2015). A dosing schedule of 4 days on in the clinical study in ALS patients (CBLZ945C12201), will be used to mitigate the risk of inducing ALT, AST and CK elevation in ALS patients. Additionally, the off period of the dosing regimen is expected to allow time for recovery of the microglia that is depleted through CSF-1R inhibition by administration of BLZ945. An additional cohort to evaluate 7 days on may also be evaluated.

One recommended dose of BLZ945 as single agent for treatment of neurodegenerative diseases, such as ALS, has been determined as 1200 mg q.d., 4 days on. Intermittent dosing that is sufficient to induce persistent microglial suppression is expected to avoid adverse effects caused by continuous dosing.

1-43. (canceled)

44. A method for treating amyotrophic lateral sclerosis in a subject in need thereof, comprising administering 4-((2-(((1R,2R)-2-hydroxycyclohexyl)amino)benzo[d]thiazol-6-yl)oxy)-N-methylpicolinamide or a pharmaceutically acceptable salt thereof to said subject for an on period of 4 days followed by an off period of up to 10 days at a daily dose of 400 mg/day, 500 mg/day, 600 mg/day, 700 mg/day, 800 mg/day, 900 mg/day, or 1200 mg/day.

45. The method of claim 44, wherein said 4-((2-(((1R,2R)-2-hydroxycyclohexyl)amino) benzo[d]thiazol-6-yl)oxy)-N-methylpicolinamide is administered for an on period of 4 days followed by an off period of up to 10 days.

46. The method of claim 44, wherein said 4-((2-(((1R,2R)-2-hydroxycyclohexyl)amino) benzo[d]thiazol-6-yl)oxy)-N-methylpicolinamide is administered at a daily dose of 600 mg/day, 800 mg/day or 1200 mg/day.

47. The method of claim 44, wherein said 4-((2-(((1R,2R)-2-hydroxycyclohexyl)amino) benzo[d]thiazol-6-yl)oxy)-N-methylpicolinamide is administered once daily.

48. The method of claim 44, wherein said 4-((2-(((1R,2R)-2-hydroxycyclohexyl)amino) benzo[d]thiazol-6-yl)oxy)-N-methylpicolinamide is administered twice daily.

49. The method of claim 44, wherein said 4-((2-(((1R,2R)-2-hydroxycyclohexyl)amino) benzo[d]thiazol-6-yl)oxy)-N-methylpicolinamide is administered at a daily dose of 600 mg/day.

50. The method of claim 44, wherein said 4-((2-(((1R,2R)-2-hydroxycyclohexyl)amino) benzo[d]thiazol-6-yl)oxy)-N-methylpicolinamide is administered at a daily dose of 800 mg/day.

51. The method of claim 44, wherein said 4-((2-(((1R,2R)-2-hydroxycyclohexyl)amino) benzo[d]thiazol-6-yl)oxy)-N-methylpicolinamide is administered at a daily dose of 1200 mg/day.

52. The method of claim 44, wherein said 4-((2-(((1R,2R)-2-hydroxycyclohexyl)amino) benzo[d]thiazol-6-yl)oxy)-N-methylpicolinamide is administered for an on period of 4 days followed by an off period of 10 days and at a daily dose of 600 mg/day, 800 mg/day or 1200 mg/day.

53. The method of claim 44, wherein said 4-((2-(((1R,2R)-2-hydroxycyclohexyl)amino) benzo[d]thiazol-6-yl)oxy)-N-methylpicolinamide is administered for an on period of 4 days followed by an off period of 10 days at a daily dose of 600 mg/day, 800 mg/day or 1200 mg/day, twice per cycle.

54. The method of claim 53, wherein said 4-((2-(((1R,2R)-2-hydroxycyclohexyl)amino) benzo[d]thiazol-6-yl)oxy)-N-methylpicolinamide is administered at a daily dose of 600 mg/day.

55. The method of claim 53 wherein said 4-((2-(((1R,2R)-2-hydroxycyclohexyl)amino) benzo[d]thiazol-6-yl)oxy)-N-methylpicolinamide is administered at a daily dose of 800 mg/day.

56. The method of claim 53, wherein said 4-((2-(((1R,2R)-2-hydroxycyclohexyl)amino) benzo[d]thiazol-6-yl)oxy)-N-methylpicolinamide is administered at a daily dose of 1200 mg/day.


SEQUENCE LISTING (225 sequences)

SEQ ID NO: 1 - protein, 5 aa, Artificial Sequence
  source: /note=“Description of Artificial Sequence: Synthetic peptide”
TYWMH

SEQ ID NO: 2 - protein, 17 aa, Artificial Sequence
  source: /note=“Description of Artificial Sequence: Synthetic peptide”
NIYPGTGGSNFDEKFKN

SEQ ID NO: 3 - protein, 8 aa, Artificial Sequence
  source: /note=“Description of Artificial Sequence: Synthetic peptide”
WTTGTGAY

SEQ ID NO: 4 - protein, 7 aa, Artificial Sequence
  source: /note=“Description of Artificial Sequence: Synthetic peptide”
GYTFTTY

SEQ ID NO: 5 - protein, 6 aa, Artificial Sequence
  source: /note=“Description of Artificial Sequence: Synthetic peptide”
YPGTGG

SEQ ID NO: 6-9 - skipped (4 entries)

SEQ ID NO: 10 - protein, 17 aa, Artificial Sequence
  source: /note=“Description of Artificial Sequence: Synthetic peptide”
KSSQSLLDSGNQKNFLT

SEQ ID NO: 11 - protein, 7 aa, Artificial Sequence
  source: /note=“Description of Artificial Sequence: Synthetic peptide”
WASTRES

SEQ ID NO: 12 - skipped

SEQ ID NO: 13 - protein, 13 aa, Artificial Sequence
  source: /note=“Description of Artificial Sequence: Synthetic peptide”
SQSLLDSGNQKNF

SEQ ID NO: 14 - protein, 3 aa, Artificial Sequence
  source: /note=“Description of Artificial Sequence: Synthetic peptide”
WAS

SEQ ID NO: 15-31 - skipped (17 entries)

SEQ ID NO: 32 - protein, 9 aa, Artificial Sequence
  source: /note=“Description of Artificial Sequence: Synthetic peptide”
QNDYSYPYT

SEQ ID NO: 33 - protein, 6 aa, Artificial Sequence
  source: /note=“Description of Artificial Sequence: Synthetic peptide”
DYSYPY

SEQ ID NO: 34-35 - skipped (2 entries)

SEQ ID NO: 36 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 39 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42-53 - skipped (12 entries)

SEQ ID NO: 54 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 55 - skipped

SEQ ID NO: 56 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57-69 - skipped (13 entries)

SEQ ID NO: 70 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 71 - skipped

SEQ ID NO: 72 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73-90 - skipped (18 entries)

SEQ ID NO: 91 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 92 - DNA, 1329 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactgggtgcgacaggctaccggccagggcctggaatggatgggcaacatctatcctggcaccggcggctccaacttcgacgagaagttcaagaacagagtgaccatcaccgccgacaagtccacctccaccgcctacatggaactgtcctccctgagatccgaggacaccgccgtgtactactgcacccggtggacaaccggcacaggcgcttattggggccagggcaccacagtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 93-94 - skipped (2 entries)

SEQ ID NO: 95 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 96 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 97 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 98 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 99-105 - skipped (7 entries)

SEQ ID NO: 106 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 107 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 108-132 - skipped (25 entries)

SEQ ID NO: 133 - DNA, 15 bp, Artificial Sequence
  source: /note=“Description of Artificial Sequence: Synthetic oligonucleotide”
acctactggatgcac

SEQ ID NO: 134 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 135 - DNA, 24 bp, Artificial Sequence
  source: /note=“Description of Artificial Sequence: Synthetic oligonucleotide”
tggactaccggcacaggcgcctac

SEQ ID NO: 136 - DNA, 21 bp, Artificial Sequence
  source: /note=“Description of Artificial Sequence: Synthetic oligonucleotide”
ggctacaccttcactacctac

SEQ ID NO: 137 - DNA, 18 bp, Artificial Sequence
  source: /note=“Description of Artificial Sequence: Synthetic oligonucleotide”
taccccggcaccggcggc

SEQ ID NO: 138 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 139 - DNA, 21 bp, Artificial Sequence
  source: /note=“Description of Artificial Sequence: Synthetic oligonucleotide”
tgggcctctactagagaatca

SEQ ID NO: 140 - DNA, 27 bp, Artificial Sequence
  source: /note=“Description of Artificial Sequence: Synthetic oligonucleotide”
cagaacgactatagctacccctacacc

SEQ ID NO: 141 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 142 - DNA, 9 bp, Artificial Sequence
  source: /note=“Description of Artificial Sequence: Synthetic oligonucleotide”
tgggcctct

SEQ ID NO: 143 - DNA, 18 bp, Artificial Sequence
  source: /note=“Description of Artificial Sequence: Synthetic oligonucleotide”
gactatagctacccctac

SEQ ID NO: 144-146 - skipped (3 entries)

SEQ ID NO: 147 - protein, 25 aa, Artificial Sequence
  source: /note=“Description of Artificial Sequence: Synthetic peptide”
EVQLVQSGAEVKKPGESLRISCKGS

SEQ ID NO: 148 - DNA, 75 bp, Artificial Sequence
  source: /note=“Description of Artificial Sequence: Synthetic oligonucleotide”
gaagtgcagctggtgcagtctggagcagaggtgaaaaagcccggggagtctctgaggatctcctgtaagggttct

SEQ ID NO: 149 - DNA, 75 bp, Artificial Sequence
  source: /note=“Description of Artificial Sequence: Synthetic oligonucleotide”
gaagtgcagctggtgcagtctggcgccgaagtgaagaagcctggcgagtccctgcggatctcctgcaagggctct

SEQ ID NO: 150 - DNA, 75 bp, Artificial Sequence
  source: /note=“Description of Artificial Sequence: Synthetic oligonucleotide”
gaggtgcagctggtgcagtcaggcgccgaagtgaagaagcccggcgagtcactgagaattagctgtaaaggttca

SEQ ID NO: 151 - protein, 25 aa, Artificial Sequence [sequence omitted]

SEQ ID NO: 152 - DNA, 75 bp, Artificial Sequence
  source: /note=“Description of Artificial Sequence: Synthetic oligonucleotide”
caggttcagctggtgcagtctggagctgaggtgaagaagcctggggcctcagtgaaggtctcctgcaaggcttct

SEQ ID NO: 153 - protein, 14 aa, Artificial Sequence
  source: /note=“Description of Artificial Sequence: Synthetic peptide”
WVRQATGQGLEWMG

SEQ ID NO: 154 - DNA, 42 bp, Artificial Sequence
  source: /note=“Description of Artificial Sequence: Synthetic oligonucleotide”
tgggtgcgacaggccactggacaagggcttgagtggatgggt

SEQ ID NO: 155 - DNA, 42 bp, Artificial Sequence
  source: /note=“Description of Artificial Sequence: Synthetic oligonucleotide”
tgggtgcgacaggctaccggccagggcctggaatggatgggc

SEQ ID NO: 156 - DNA, 42 bp, Artificial Sequence
  source: /note=“Description of Artificial Sequence: Synthetic oligonucleotide”
tgggtccgccaggctaccggtcaaggcctcgagtggatgggt

SEQ ID NO: 157 - protein, 14 aa, Artificial Sequence
  source: /note=“Description of Artificial Sequence: Synthetic peptide”
WIRQSPSRGLEWLG

SEQ ID NO: 158 - DNA, 42 bp, Artificial Sequence
  source: /note=“Description of Artificial Sequence: Synthetic oligonucleotide”
tggatcaggcagtccccatcgagaggccttgagtggctgggt

SEQ ID NO: 159 - DNA, 42 bp, Artificial Sequence
  source: /note=“Description of Artificial Sequence: Synthetic oligonucleotide”
tggatccggcagtccccctctaggggcctggaatggctgggc

SEQ ID NO: 160 - protein, 14 aa, Artificial Sequence
  source: /note=“Description of Artificial Sequence: Synthetic peptide”
WVRQAPGQGLEWMG

SEQ ID NO: 161 - DNA, 42 bp, Artificial Sequence
  source: /note=“Description of Artificial Sequence: Synthetic oligonucleotide”
tgggtgcgacaggcccctggacaagggcttgagtggatgggt

SEQ ID NO: 162 - protein, 32 aa, Artificial Sequence
  source: /note=“Description of Artificial Sequence: Synthetic polypeptide”
RVTITADKSTSTAYMELSSLRSEDTAVYYCTR

SEQ ID NO: 163 - DNA, 96 bp, Artificial Sequence
  source: /note=“Description of Artificial Sequence: Synthetic oligonucleotide”
agagtcacgattaccgcggacaaatccacgagcacagcctacatggagctgagcagcctgagatctgaggacacggccgtgtattactgtacaaga

SEQ ID NO: 164 - DNA, 96 bp, Artificial Sequence
  source: /note=“Description of Artificial Sequence: Synthetic oligonucleotide”
agagtgaccatcaccgccgacaagtccacctccaccgcctacatggaactgtcctccctgagatccgaggacaccgccgtgtactactgcacccgg

SEQ ID NO: 165 - DNA, 96 bp, Artificial Sequence
  source: /note=“Description of Artificial Sequence: Synthetic oligonucleotide”
agagtgactatcaccgccgataagtctactagcaccgcctatatggaactgtctagcctgagatcagaggacaccgccgtctactactgcactagg

SEQ ID NO: 166 - protein, 32 aa, Artificial Sequence
  source: /note=“Description of Artificial Sequence: Synthetic polypeptide”
RFTISRDNSKNTLYLQMNSLRAEDTAVYYCTR

SEQ ID NO: 167 - DNA, 96 bp, Artificial Sequence
  source: /note=“Description of Artificial Sequence: Synthetic oligonucleotide”
agattcaccatctccagagacaattccaagaacacgctgtatcttcaaatgaacagcctgagagccgaggacacggccgtgtattactgtacaaga

SEQ ID NO: 168 - DNA, 96 bp, Artificial Sequence
  source: /note=“Description of Artificial Sequence: Synthetic oligonucleotide”
aggttcaccatctcccgggacaactccaagaacaccctgtacctgcagatgaactccctgcgggccgaggacaccgccgtgtactactgtaccaga

SEQ ID NO: 169 - protein, 11 aa, Artificial Sequence
  source: /note=“Description of Artificial Sequence: Synthetic peptide”
WGQGTTVTVSS

SEQ ID NO: 170 - DNA, 33 bp, Artificial Sequence
  source: /note=“Description of Artificial Sequence: Synthetic oligonucleotide”
tggggccagggcaccaccgtgaccgtgtcctcc

SEQ ID NO: 171 - DNA, 33 bp, Artificial Sequence
  source: /note=“Description of Artificial Sequence: Synthetic oligonucleotide”
tggggccagggcaccacagtgaccgtgtcctct

SEQ ID NO: 172 - DNA, 33 bp, Artificial Sequence
  source: /note=“Description of Artificial Sequence: Synthetic oligonucleotide”
tggggtcaaggcactaccgtgaccgtgtctagc

SEQ ID NO: 173 - DNA, 33 bp, Artificial Sequence
  source: /note=“Description of Artificial Sequence: Synthetic oligonucleotide”
tggggccagggcacaacagtgaccgtgtcctcc

SEQ ID NO: 174 - protein, 23 aa, Artificial Sequence
  source: /note=“Description of Artificial Sequence: Synthetic peptide”
EIVLTQSPDFQSVTPKEKVTITC

SEQ ID NO: 175 - DNA, 69 bp, Artificial Sequence
  source: /note=“Description of Artificial Sequence: Synthetic oligonucleotide”
gaaattgtgctgactcagtctccagactttcagtctgtgactccaaaggagaaagtcaccatcacctgc

SEQ ID NO: 176 - DNA, 69 bp, Artificial Sequence
  source: /note=“Description of Artificial Sequence: Synthetic oligonucleotide”
gagatcgtgctgacccagtcccccgacttccagtccgtgacccccaaagaaaaagtgaccatcacatgc

SEQ ID NO: 177 - protein, 23 aa, Artificial Sequence
  source: /note=“Description of Artificial Sequence: Synthetic peptide”
EIVLTQSPATLSLSPGERATLSC

SEQ ID NO: 178 - DNA, 69 bp, Artificial Sequence
  source: /note=“Description of Artificial Sequence: Synthetic oligonucleotide”
gaaattgtgttgacacagtctccagccaccctgtctttgtctccaggggaaagagccaccctctcctgc

SEQ ID NO: 179 - DNA, 69 bp, Artificial Sequence
  source: /note=“Description of Artificial Sequence: Synthetic oligonucleotide”
gagatcgtgctgacccagtcccctgccaccctgtcactgtctccaggcgagagagctaccctgtcctgc

SEQ ID NO: 180 - DNA, 69 bp, Artificial Sequence
  source: /note=“Description of Artificial Sequence: Synthetic oligonucleotide”
gagatcgtcctgactcagtcacccgctaccctgagcctgagccctggcgagcgggctacactgagctgt

SEQ ID NO: 181 - protein, 23 aa, Artificial Sequence
  source: /note=“Description of Artificial Sequence: Synthetic peptide”
DIVMTQTPLSLPVTPGEPASISC

SEQ ID NO: 182 - DNA, 69 bp, Artificial Sequence
  source: /note=“Description of Artificial Sequence: Synthetic oligonucleotide”
gatattgtgatgacccagactccactctccctgcccgtcacccctggagagccggcctccatctcctgc

SEQ ID NO: 183 - protein, 23 aa, Artificial Sequence
  source: /note=“Description of Artificial Sequence: Synthetic peptide”
DVVMTQSPLSLPVTLGQPASISC

SEQ ID NO: 184 - DNA, 69 bp, Artificial Sequence
  source: /note=“Description of Artificial Sequence: Synthetic oligonucleotide”
gatgttgtgatgactcagtctccactctccctgcccgtcacccttggacagccggcctccatctcctgc

SEQ ID NO: 185 - protein, 23 aa, Artificial Sequence
  source: /note=“Description of Artificial Sequence: Synthetic peptide”
DIQMTQSPSSLSASVGDRVTITC

SEQ ID NO: 186 - DNA, 69 bp, Artificial Sequence
  source: /note=“Description of Artificial Sequence: Synthetic oligonucleotide”
gacatccagatgacccagtctccatcctccctgtctgcatctgtaggagacagagtcaccatcacttgc

SEQ ID NO: 187 - protein, 15 aa, Artificial Sequence
  source: /note=“Description of Artificial Sequence: Synthetic peptide”
WYQQKPGQAPRLLIY

SEQ ID NO: 188 - DNA, 45 bp, Artificial Sequence
  source: /note=“Description of Artificial Sequence: Synthetic oligonucleotide”
tggtaccagcagaaacctggccaggctcccaggctcctcatctat

SEQ ID NO: 189 - DNA, 45 bp, Artificial Sequence
  source: /note=“Description of Artificial Sequence: Synthetic oligonucleotide”
tggtatcagcagaagcccggccaggcccccagactgctgatctac

SEQ ID NO: 190 - DNA, 45 bp, Artificial Sequence
  source: /note=“Description of Artificial Sequence: Synthetic oligonucleotide”
tggtatcagcagaagcccggtcaagcccctagactgctgatctac

SEQ ID NO: 191 - protein, 15 aa, Artificial Sequence
  source: /note=“Description of Artificial Sequence: Synthetic peptide”
WYQQKPGKAPKLLIY

SEQ ID NO: 192 - DNA, 45 bp, Artificial Sequence
  source: /note=“Description of Artificial Sequence: Synthetic oligonucleotide”
tggtatcagcagaaaccagggaaagctcctaagctcctgatctat

SEQ ID NO: 193 - DNA, 45 bp, Artificial Sequence
  source: /note=“Description of Artificial Sequence: Synthetic oligonucleotide”
tggtatcagcagaagcccggtaaagcccctaagctgctgatctac

SEQ ID NO: 194 - protein, 15 aa, Artificial Sequence
  source: /note=“Description of Artificial Sequence: Synthetic peptide”
WYLQKPGQSPQLLIY

SEQ ID NO: 195 - DNA, 45 bp, Artificial Sequence
  source: /note=“Description of Artificial Sequence: Synthetic oligonucleotide”
tggtacctgcagaagccagggcagtctccacagctcctgatctat

SEQ ID NO: 196 - protein, 32 aa, Artificial Sequence
  source: /note=“Description of Artificial Sequence: Synthetic polypeptide”
GVPSRFSGSGSGTDFTFTISSLEAEDAATYYC

SEQ ID NO: 197 - DNA, 96 bp, Artificial Sequence
  source: /note=“Description of Artificial Sequence: Synthetic oligonucleotide”
ggggtcccctcgaggttcagtggcagtggatctgggacagatttcacctttaccatcagtagcctggaagctgaagatgctgcaacatattactgt

SEQ ID NO: 198 - DNA, 96 bp, Artificial Sequence
  source: /note=“Description of Artificial Sequence: Synthetic oligonucleotide”
ggcgtgccctctagattctccggctccggctctggcaccgactttaccttcaccatctccagcctggaagccgaggacgccgccacctactactgc

SEQ ID NO: 199 - DNA, 96 bp, Artificial Sequence
  source: /note=“Description of Artificial Sequence: Synthetic oligonucleotide”
ggcgtgccctctaggtttagcggtagcggtagtggcaccgacttcaccttcactatctctagcctggaagccgaggacgccgctacctactactgt

SEQ ID NO: 200 - protein, 32 aa, Artificial Sequence
  source: /note=“Description of Artificial Sequence: Synthetic polypeptide”
GIPPRFSGSGYGTDFTLTINNIESEDAAYYFC

SEQ ID NO: 201 - DNA, 96 bp, Artificial Sequence
  source: /note=“Description of Artificial Sequence: Synthetic oligonucleotide”
gggatcccacctcgattcagtggcagcgggtatggaacagattttaccctcacaattaataacatagaatctgaggatgctgcatattacttctgt

SEQ ID NO: 202 - protein, 32 aa, Artificial Sequence
  source: /note=“Description of Artificial Sequence: Synthetic polypeptide”
GVPSRFSGSGSGTEFTLTISSLQPDDFATYYC

SEQ ID NO: 203 - DNA, 96 bp, Artificial Sequence
  source: /note=“Description of Artificial Sequence: Synthetic oligonucleotide”
ggggtcccatcaaggttcagcggcagtggatctgggacagaattcactctcaccatcagcagcctgcagcctgatgattttgcaacttattactgt

SEQ ID NO: 204 - DNA, 96 bp, Artificial Sequence
  source: /note=“Description of Artificial Sequence: Synthetic oligonucleotide”
ggcgtgccctctagattctccggctccggctctggcaccgagtttaccctgaccatctccagcctgcagcccgacgacttcgccacctactactgc

SEQ ID NO: 205 - protein, 32 aa, Artificial Sequence
  source: /note=“Description of Artificial Sequence: Synthetic polypeptide”
GVPSRFSGSGSGTDFTFTISSLQPEDIATYYC

SEQ ID NO: 206 - DNA, 96 bp, Artificial Sequence
  source: /note=“Description of Artificial Sequence: Synthetic oligonucleotide”
ggggtcccatcaaggttcagtggaagtggatctgggacagattttactttcaccatcagcagcctgcagcctgaagatattgcaacatattactgt

SEQ ID NO: 207 - DNA, 96 bp, Artificial Sequence
  source: /note=“Description of Artificial Sequence: Synthetic oligonucleotide”
ggcgtgccctctaggtttagcggtagcggtagtggcaccgacttcaccttcactatctctagcctgcagcccgaggatatcgctacctactactgt

SEQ ID NO: 208 - protein, 10 aa, Artificial Sequence
  source: /note=“Description of Artificial Sequence: Synthetic peptide”
FGQGTKVEIK

SEQ ID NO: 209 - DNA, 30 bp, Artificial Sequence
  source: /note=“Description of Artificial Sequence: Synthetic oligonucleotide”
ttcggccaagggaccaaggtggaaatcaaa

SEQ ID NO: 210 - DNA, 30 bp, Artificial Sequence
  source: /note=“Description of Artificial Sequence: Synthetic oligonucleotide”
ttcggccagggcaccaaggtggaaatcaag

SEQ ID NO: 211 - DNA, 30 bp, Artificial Sequence
  source: /note=“Description of Artificial Sequence: Synthetic oligonucleotide”
ttcggtcaaggcactaaggtcgagattaag

SEQ ID NO: 212 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 107 aa, Homo sapiens [sequence omitted]

SEQ ID NO: 214 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 215 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219-220 - skipped (2 entries)

SEQ ID NO: 221 - protein, 19 aa, Artificial Sequence
  source: /note=“Description of Artificial Sequence: Synthetic peptide”
MAWVWTLPFLMAAAQSVQA

SEQ ID NO: 222 - protein, 20 aa, Artificial Sequence
  source: /note=“Description of Artificial Sequence: Synthetic peptide”
MSVLTQVLALLLLWLTGTRC

SEQ ID NO: 223 - skipped

SEQ ID NO: 224 - protein, 10 aa, Artificial Sequence
  source: /note=“Description of Artificial Sequence: Synthetic peptide”
GYTFTTYWMH

SEQ ID NO: 225 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK